(19) United States
(12) Patent Application Publication
(10) Pub. No.: US 2023/0170059 A1
(43) Pub. Date: Jun. 1, 2023

(54) MOLECULAR STRUCTURE TRANSFORMERS FOR PROPERTY PREDICTION

(71) Applicant: X Development LLC, Mountain View, CA (US)

(72) Inventors: Tusharkumar Gadhiya, Gandhinagar (IN); Falak Shah, Mountain View, CA (US); Nisarg Vyas, Mountain View, CA (US); Julia Yang, Berkeley, CA (US); Vahe Gharakhanyan, Mountain View, CA (US); Alexander Holiday, Brookfield, WI (US)

(21) Appl. No.: 17/967,685

(22) Filed: Oct. 17, 2022

Related U.S. Application Data
(60) Provisional application No. 63/264,641, filed on Nov. 29, 2021.
(60) Provisional application No. 63/264,642, filed on Nov. 29, 2021.
(60) Provisional application No. 63/264,643, filed on Nov. 29, 2021.
(60) Provisional application No. 63/264,640, filed on Nov. 29, 2021.

(51) Int. Cl. G16C 20/70; G16C 20/20; G16C 20/80

(52) U.S. Cl. CPC G16C 20/70; G16C 20/20; G16C 20/80

(57) ABSTRACT

Computer-implemented methods may include accessing a multi-dimensional embedding space that supports relating embeddings of molecules to predicted values of a given property of the molecules. The method may also include identifying one or more points of interest within the embedding space based on the predicted values. Each of the one or more points of interest may include a set of coordinate values within the multi-dimensional embedding space and may be associated with a corresponding predicted value of the given property. The method may further include generating, for each of the one or more points of interest, a structural representation of a molecule by transforming the set of coordinate values included in the point of interest using a decoder network. The method may include outputting a result that identifies, for each of the one or more points of interest, the structural representation of the molecule corresponding to the point of interest.

### CROSS-REFERENCE TO RELATED APPLICATIONS

This application is a non-provisional of and claims the benefit of U.S. Provisional Application No. 63/264,640, filed Nov. 29, 2021; U.S. Provisional Application No. 63/264,641, filed Nov. 29, 2021; U.S. Provisional Application No. 63/264,642, filed Nov. 29, 2021; and U.S. Provisional Application No. 63/264,643, filed Nov. 29, 2021; the contents of all of which are incorporated herein by reference in their entirety for all purposes.

### BACKGROUND

A problem in chemistry is predicting a certain property of some new molecule. Predicting properties of molecules is useful in identifying new molecules to use in recycling. Chemical recycling aims to break down plastic waste into the monomeric building blocks it was produced from, enabling a circular economy in which polymers are produced from chemically recycled plastics instead of relying on nonrenewable inputs derived from petroleum. Plastic recycling may include the conversion of waste plastics (polyethylene terephthalate (PET), polylactic acid (PLA)) into their monomer components (bis(2-hydroxyethyl) terephthalate (BHET), lactate) to replace virgin plastics derived from oil. Ionic liquids (ILs) are a highly tunable class of chemicals that has shown a promising ability to depolymerize plastics, but it's unclear how to navigate the large ionic-liquid design space to improve reaction yields.

Selecting a specific ionic liquid to use for depolymerization is a challenging task. First, given the number of ionic liquid candidates and different reaction conditions that exist, experimentally characterizing properties of all ionic liquids in pertinent conditions is infeasible. More specifically, ionic liquids consist of tunable choices of cation and anion molecules, leading to a high-dimensional space from which to choose the experimental parameters. For example, in the National Institute of Standards & Technology (NIST) ILThermo database, there are 1,652 binary ILs with 244 cations and 164 anions. Combinatorially, this means there are 38,364 additional new ILs to be generated from the NIST database alone. Choosing a specific IL under a sampling of experimental conditions (such as exploring 3 solvents, 5 ratios of ionic liquids to solvent, 3 temperatures, and 3 reaction time periods) results in a vastly complex reaction space containing over 5,400,000 different reaction conditions. In a typical design of experiment, domain knowledge and literature review are requirements to reduce the search space, but this process is costly and does not lend itself to an evaluation of the complete design space.

Thus, being able to better predict properties of ionic liquid molecules and/or results of reactions involving ionic liquid molecules may facilitate more efficient recycling.

One approach for generating these predictions is to use machine learning to transform a representation of the new molecule into a prediction. However, machine learning requires that the molecule then be represented by a set of numbers (e.g., via featurization, fingerprinting, or embedding).

However, existing techniques for numerically representing molecules fail to capture complete structural information of the molecule. Rather, the structural information is either neglected entirely or only partially represented.

### BRIEF SUMMARY

Some embodiments may include a computer-implemented method. The method may include accessing a multi-dimensional embedding space that supports relating embeddings of molecules to predicted values of a given property of the molecules. The method may also include identifying one or more points of interest within the multi-dimensional embedding space based on the predicted values. Each of the one or more points of interest may include a set of coordinate values within the multi-dimensional embedding space, may convey spatial information of the atoms or bonds in the molecule, and may be associated with a corresponding predicted value of the given property. The method may further include generating, for each of the one or more points of interest, a structural representation of a molecule by transforming the set of coordinate values included in the point of interest using a decoder network. Training of the decoder network may have included learning to transform positions within the embedding space to outputs representing molecular-structure characteristics. Training of the decoder network may have been performed at least in part concurrently to training an encoder network. The method may include outputting a result that identifies, for each of the one or more points of interest, the structural representation of the molecule corresponding to the point of interest.

In some embodiments, training of the encoder network may have included learning to transform partial or complete bond string and position (BSP) representations of molecules into positions within the embedding space. Each BSP representation may identify relative positions of atoms connected by a bond in the represented molecule.

In some embodiments, training of the encoder network may have included learning to transform partial or complete molecular graph representations of molecules into positions within the embedding space. Each molecular graph representation may identify angles and distances of bonds in the represented molecule.

In some embodiments, the decoder network and the encoder network may be trained by training a transformer model that uses self-attention. The transformer model may include the decoder network and the encoder network.

In some embodiments, the decoder network and the encoder network may be trained by training a transformer model that includes an attention head.

In some embodiments, the method may include training a machine-learning model that includes the encoder network and the decoder molecule by accessing a set of supplemental training elements. Each of the set of training elements may include a representation of a structure of a corresponding given molecule. The training may further include masking, for each supplemental training element in the set of supplemental training elements, at least part of the representation to obscure at least part of the structure of the corresponding given molecule. The training may include training the machine-learning model to predict the obscured at least part of the structure.

In some embodiments, training of the encoder network further may include fine-tuning the encoder network to transform positions within the space to predictions corresponding to values of the given property.

In some embodiments, each BSP representation of the molecules used to train the encoder network may include a set of coordinates for each of the atoms connected by the bond in the represented molecule and may further identify each of the atoms connected by the bond in the represented molecule.

In some embodiments, the BSP representation of the molecules may be used to train the encoder network to identify, for each of at least some bonds in a respective molecule, a bond type.

In some embodiments, a format of the structural representation identified in the result may be different than the BSP representation.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIGS. 1A, 1B, 1C, 1D, and 1E show a pictorial representation of the process of generating the SMILES string for ciprofloxacin: C1CC1N2C═C(C(═O)C3=CC(═C(C═C32)N4CCNCC4)F)C(═O)O according to embodiments of the present invention.

FIG. 2 depicts an illustrative process for construction of BSP representation of a single molecule from SMILES strings according to embodiments of the present invention.

FIG. 3 depicts an illustrative process for construction of BSP representations of reactant/reagents from SMILES strings according to embodiments of the present invention.

FIG. 4 depicts an illustrative process for generating a token embedding that is an input to a model (e.g., a Transformer model) according to embodiments of the present invention.

FIG. 5 shows a depiction of masked bond model training in the context of molecular property prediction according to embodiments of the present invention. The input molecule is shown at the bottom with masked bonds indicated in black. This is mapped to a BSP representation, replacing masked bonds with the special mask token, and fed to the Encoder network. The model returns a prediction of the bond at each point, eventually learning to accurately fill in the masked locations.

FIG. 6 depicts an illustrative use of Encoder and Decoder networks to transform representations between structural identifiers and an embedding space according to embodiments of the present invention.

FIG. 7 shows a depiction of a message passing graph convolution layer according to embodiments of the present invention.

FIG. 8 illustrates how, in Atom Masking, the nodes are randomly masked, and the GNN is trained to predict the correct labels of masked nodes. Figure is based on Hu et al., arXiv: 1905.12265v3.

FIG. 9 illustrates how, in context prediction, the subgraph is a K-hop neighborhood around a selected center node, where K is the number of GNN layers. Figure is based on Hu et al., arXiv: 1905.12265v3.

FIG. 10 illustrates an objective of a latent representation of a molecule according to embodiments of the present invention.

FIG. 11 illustrates computation of relative position in terms of angles and distances according to embodiments of the present invention.

FIGS. 12A and 12B illustrate message generation and message aggregation according to embodiments of the present invention.

FIG. 13 illustrates readout attention aggregation according to embodiments of the present invention.

FIG. 14 illustrates an overview of the model that includes the Encoder network used for molecular property prediction according to embodiments of the present invention.

FIG. 15 illustrates a reaction prediction model according to embodiments of the present invention. The main component is a Transformer-based architecture that operates on BSP inputs and returns a SMILES string predicting the reaction products.

FIGS. 16A and 16B show examples of correctly predicted reactions showing the model's understanding of a reaction's active sites according to embodiments of the present invention.

FIGS. 17A and 17B show examples of incorrectly predicted reactions.

FIG. 18 shows attention weights of the Encoder network's fourth head in its third layer according to embodiments of the present invention.

FIG. 19 illustrates traversing a molecular graph in a depth-first search to construct the bond string representation according to embodiments of the present invention.

FIG. 20 illustrates the Directional Variational Transformer model according to embodiments of the present invention.

FIG. 21 illustrates relationships between molecules, embeddings, and property predictions according to embodiments of the present invention.

FIG. 22 illustrates a process for identifying ionic liquids to depolymerize a compound according to embodiments of the present invention.

FIG. 23 shows ionic liquid cations generated from exploring embedding space around a molecule according to embodiments of the present invention.

FIG. 24 shows ionic liquid cations generated from exploring embedding space between two molecules according to embodiments of the present invention.

FIG. 25 illustrates the interaction between Bayesian optimization and benchtop experiments according to embodiments of the present invention.

FIG. 26 illustrates a Bayesian optimization process according to embodiments of the present invention.

FIG. 27 shows a flowchart of an example process associated with ionic liquid based depolymerization optimization according to embodiments of the present invention.

FIGS. 28A and 28B show the result of applying Bayesian Optimization to the minimization of enthalpy of mixing according to embodiments of the present invention.

FIG. 29 is an illustrative architecture of a computing system implemented as some embodiments of the present disclosure.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

An embedding framework can map individual molecules into embeddings in a high dimensional space in which structurally similar molecules are mapped closer together. These representations can be processed using molecular property prediction models, and novel molecules can be identified within a space corresponding to the representations from some seed set of interest. These embeddings can be fed as input to a model that estimates certain thermodynamic properties that can be used to predict a molecule's ability to break down a certain polymer. Molecules with unfavorable properties can be filtered out, a search can be expanded around the promising candidates, eventually returning a small set of molecules (e.g., ionic liquids) that are predicted to efficiently depolymerize plastics. Candidate molecules can be processed by a Bayesian optimization system that recommends new experiments, learns from their results, and recommends further experiments until converging to optimal reaction performance. The Bayesian optimization may also be performed over a learned embedding space using the embedding framework.

### II. Machine Learning for Generating Multi-Dimensional Representations of Molecules

Accurately representing a molecule is key for predicting properties of the molecule using a model, designing novel molecules with desired properties or predicting chemical reaction outputs. Existing approaches for representing a molecule include two categories: property-based and model-based.

A property-based fingerprint is a collection of features describing different aspects of the molecule. For example, a molecule can be represented by a vector describing the number of atoms of each type it contains as shown for methanol below.

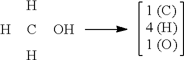

An example property-based fingerprint of methanol may include the count of each atom in the molecule.

Another example of a property-based fingerprint is the Morgan fingerprint. The Morgan fingerprint (sometimes known as the extended connectivity fingerprint, ECFP) uses limited structural information to build up a vector representation for a molecule. In particular, the Morgan fingerprint is limited by only partially capturing a molecule's structure while not accounting for the three-dimensional orientation of the atoms. And while fingerprints capture some detail of a molecule's structure, they are fundamentally limited by the availability of chemical data since each property included in the fingerprint must be included for every molecule in the dataset. In general, there is a paucity of experimental chemistry data suitable for machine learning. Note that the Morgan fingerprint does not include any explicit property information beyond an approximate encoding of the molecular graph, allowing it to be easily applied to any molecule, which contributes to its widespread use.

Model-based fingerprints rely on machine learning to generate these vector representations and include two classes: deep neural networks (DNNs) and graph neural networks (GNNs). GNNs capture molecular structure by operating directly on the molecular graph but are computationally limited in their ability to capture long-range interactions within a molecule. The molecular graph (i.e., chemical graph) is a representation of the structural formula of a molecule. The graph may include vertices corresponding to atoms and edges corresponding to bonds. DNNs may be more flexible, but they generally treat molecules as text by using string representations as input. The most popular of these string representations is SMILES, to a lesser extent, SELF-referencing Embedded Strings (SELFIES). These representations are typically obtained by traversing molecular graphs in a depth-first search (i.e., an algorithm for visiting every node in a graph) and using tokens to represent rings and branch structures.

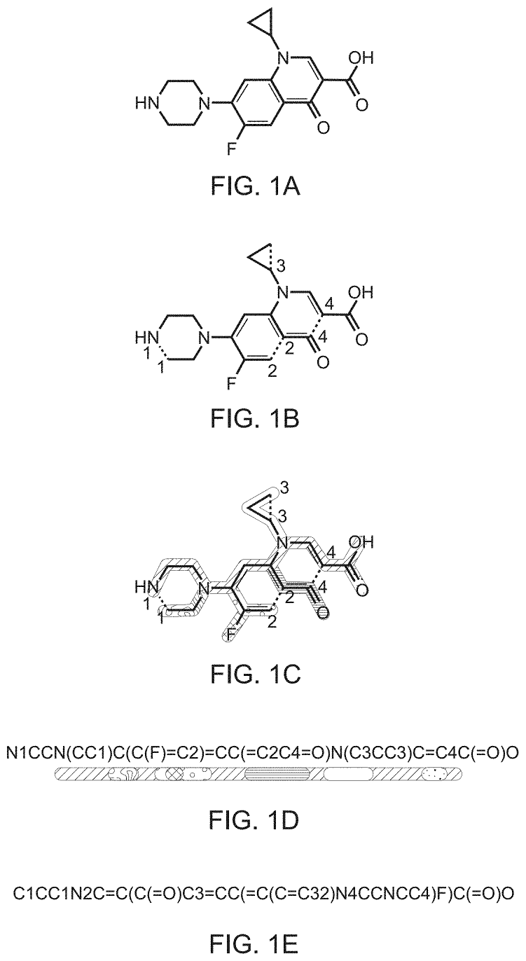

FIGS. 1A-1E illustrate a process of generating a SMILES string for a particular molecule. FIG. 1A shows an example of a molecular graph. FIG. 1B shows where cyclic structures are broken so that the molecule can be written as a string. FIG. 1C shows highlighting of various components of the molecule. FIG. 1D shows a SMILES string corresponding to the highlighting in FIG. 1C. FIG. 1E shows another SMILES string corresponding the molecular graph in FIG. 1A.

Certain approaches have represented molecules as text and applied techniques from the field of natural language processing (NLP) to, for example, predict products given reactants. But while string representations are flexible enough to describe any arbitrary molecule, they may fail to capture the rich three-dimensional structure of a molecule. For example, in FIG. 1E, the fluorine atom, F, is a significant spatial distance from the carboxylic acid group, C(═O)O, but is nearly adjacent in the SMILES string. Additionally, as shown in FIGS. 1D and 1E, a single molecule may be represented by more than one SMILES string.

Limited information content of these string representations may explain why previous NLP-inspired models do not perform well on property prediction tasks.

Embodiments described herein include encoding molecules into an embedding space. The embedding space may convey spatial information of the atoms or bonds in the molecule. For example, an encoder network may transform partial or complete bond string and position (BSP) representations, which may include coordinates of atoms, into positions within the embedding space. As another example, an encoder network may transform a molecular graph representation of a molecule into positions within the embedding space. The molecular graph representation may include angles and distances of atoms or bonds in the molecule, possibly relative to other atoms.

II.A. Bond String and Position Molecular Representations

Thus, in some embodiments of the present invention, three-dimensional information of molecules may be represented through a bond string and position (BSP) molecular representation that simultaneously captures both the chemical makeup (bond string) and the three-dimensional structure (bond position) of an arbitrary molecule. The BSP molecular representation can be generated using (for example) RDKit's structure optimization methods, which can identify three-dimensional coordinates of each atom in the molecule. Other models that can identify three-dimensional coordinates may also be used. For example, a connection table can be transformed into a distance bounds matrix, which can be smoothed using a triangle-bounds smoothing algorithm. The smoothed bounds matrix can be used to identify a conforming random distance matrix, which can be embedded in three dimensions, so as to identify three-dimensional coordinates for each atom in the molecule. A crude force field and the bounds matrix can be used to fine-tune the atoms' coordinates. As another example, instead of fine-tuning coordinates using the crude force field and bound matrix, torsion angle preferences from the Cambridge Structural Database can be used to fine-tune the coordinates. For instance, the experimental-torsion basic knowledge distance geometry (ETKDG) approach can be used to identify three-dimensional coordinates for each atom in the molecule.

Each bond in a molecule can then be represented as: <first atom><bond type><second atom> (e.g., “C10O” for a carbon atom attached to an oxygen atom via a single bond), and its corresponding bond position is represented by: [<coordinates of first atom>, <coordinates of second atom>]. This representation does not require any tokens to specify branches and rings because this information is inherently present in the coordinates of each bond. That is, the molecule's three-dimensional structure can be directly included in the model inputs, instead of requiring the model to learn this structure from a SMILES string.

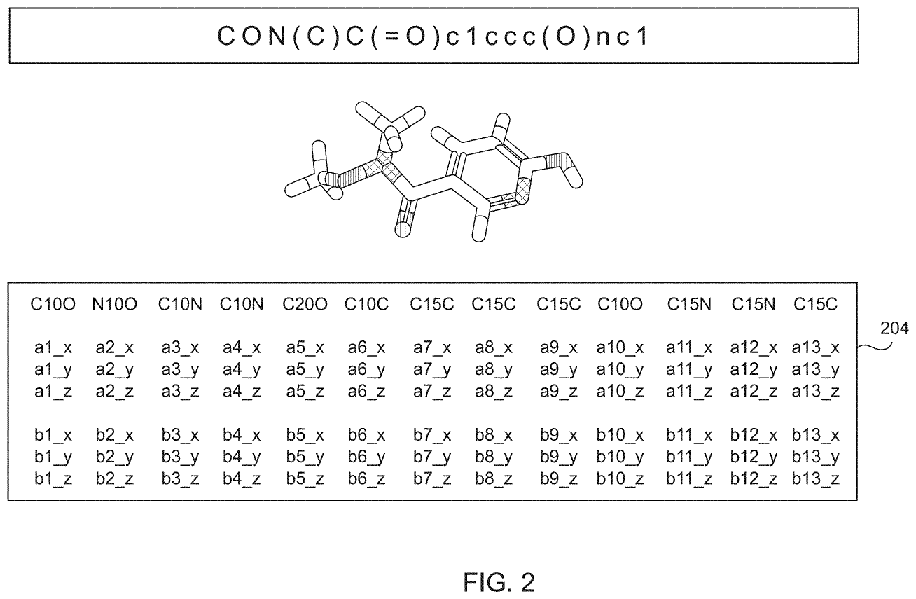

FIG. 2 shows an example of a BSP representation. The top of FIG. 2 shows a SMILES string above a molecular graph. Table 204 shows the BSP representation. The first row of table 204 shows the representation of the bond as a string token. The entries in the same column under the bond representation show the coordinates of the first atom and the second atom of each bond. The coordinates of the first atom are denoted with an “a”, and the coordinates of the second atom are denoted with a “b”. The x, y, and z denote the three-dimensional coordinate system.

II.B. Reactant/Reagent Sequence Representations

In the BSP representations, bond positions directly capture positional information. Therefore, separate token-level positional embeddings need not be used to identify bond positions, in contrast to standard Transformer-type models. However, to distinguish between distinct molecules in a single reaction, a static “molecule position” embedding can be used to indicate to which molecule in a reactant/reagent sequence the bond corresponds. Thus, the unique position of any bond in a reaction sequence can be defined by the bond position and the molecule position.

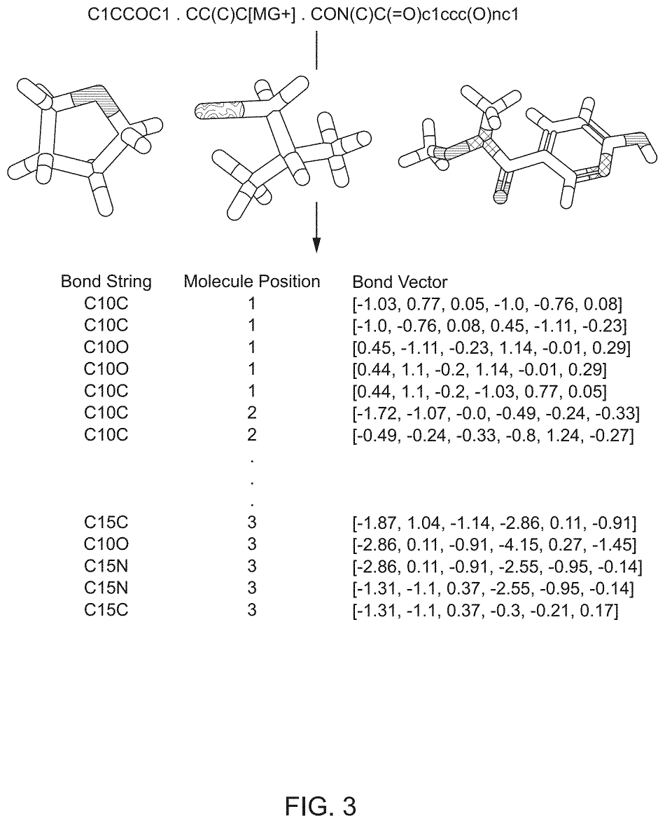

FIG. 3 shows an example of constructing BSP representations of reactants/reagents from SMILES strings. The bond strings shown in the left column enumerate the bonds in each molecule. The molecule position in the center column indicates which molecule a bond belongs to, allowing the model to distinguish one molecule from another. The third column, bond vector, contains the coordinates of the bond in three-dimensional space.

II.C. Transformer Model for Generating Fingerprints of Molecules

The BSP representations of molecules can be used as input to an Encoder network to transform the representations into embedded representations in an embedding space. The Encoder network can be pre-trained by training a machine learning model that includes the Encoder network to perform a given task. The machine learning network can include a Transformer network, including a BERT (Bidirectional Encoder Representations from Transformers) model. The given task can include predicting specifics of masked bonds within molecules. For example, a BERT model may be trained to predict a missing bond token from an incomplete initial bond string representation of a molecule.

The dimensionality of the embedding space may be smaller than the dimensionality of BSP representations. The embedding space may be a high-dimensional embedding space having at least 3 dimensions, at least 5 dimensions, at least 10 dimensions, at least 20 dimensions, at least 30 dimensions, or at least 50 dimensions. The embedding space may alternatively or additionally have fewer than 5 dimensions, fewer than 10 dimensions, fewer than 20 dimensions, fewer than 30 dimensions, fewer than 50 dimensions, or fewer than 70 dimensions. Within the embedding space, molecules that are structurally similar may be separated by small distances, while molecules lacking structural similarity may be separated by large distances. The BSP representations that are input to the Transformer model can include one, two, or three of the following embeddings:
1. Standard learnable embedding used for each token in a bond string;
2. A bond position embedding, which is obtained by projecting the bond position into the same dimension as the bond embedding, encodes the position of a bond within a molecule;
3. A molecule position embedding, which is a positional embedding used in a Transformer based model. All bonds belonging to the same molecule have the same molecule position embedding.

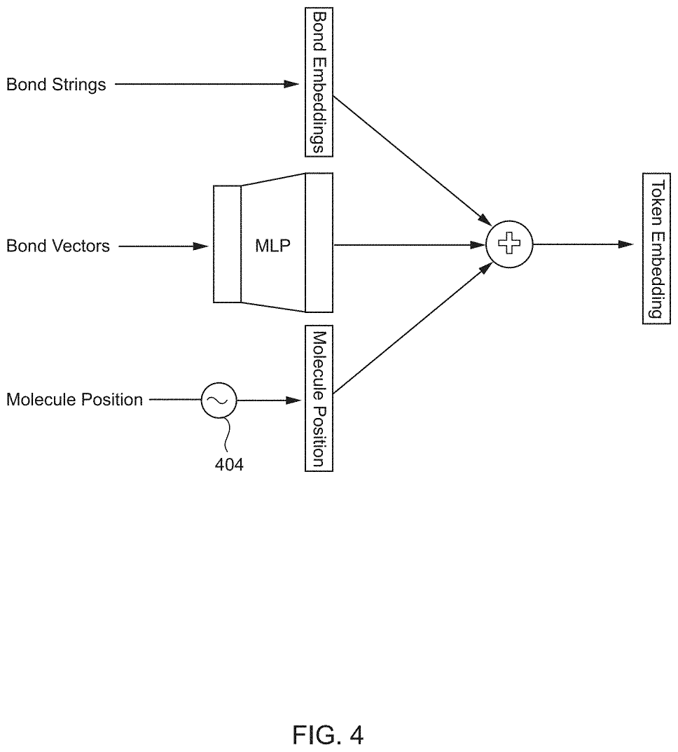

For example, FIG. 4 illustrates a token embedding (that can then be fed to a Transformer model) that includes a combination of all three of the listed embeddings. Bond embeddings are determined from the bond strings. Bond position embeddings are obtained from bond vectors using a neural network layer (e.g., MLP [Multi-Layer Perception]). Molecular position embeddings are obtained from the molecule positions. Item 404 indicates static sinusoidal embedding, which is a fixed vector that helps distinguish different molecules in this embodiment. The bond embeddings, bond position embeddings, and molecule position embeddings make up the token embedding.

II.D. Pre-Training the Transformer Model

Pre-training the Transformer model as a variational autoencoder may generate fingerprints such that molecules that are more structurally similar have fingerprints that are closer together. These fingerprints, in turn, can be used on a diverse range of tasks, from thermodynamic property prediction and toxicity classification, to achieve state of the art performance. The model may outperform several other models in property prediction.

The bond string and position (BSP) molecular representation can directly identify information about a molecule's complete three-dimensional structure. The BSP molecular representations can be used to train a machine learning model (e.g., a Transformer based model). For example, the model may be trained to predict “missing” (or “masked”) bonds that were artificially removed from each representation based on the rest of the representation. That is, the model is trained to, given the position of an unknown bond, predict the correct bond token by examining the neighboring bonds in three-dimensional space.

A training data set can include three-dimensional representations of molecules. For example, a training data set may include unique molecules from the MIT_USPTO dataset, which contains hundreds of thousands of chemical reactions scraped from U.S. patents, for a total of around 600 k unique molecules. Since the three-dimensional representation of each molecule is not unique as a single molecule may have multiple conformers, multiple molecule representations can be generated with different coordinates. This acts as a data augmentation routine and helps reduce overfitting on downstream tasks.

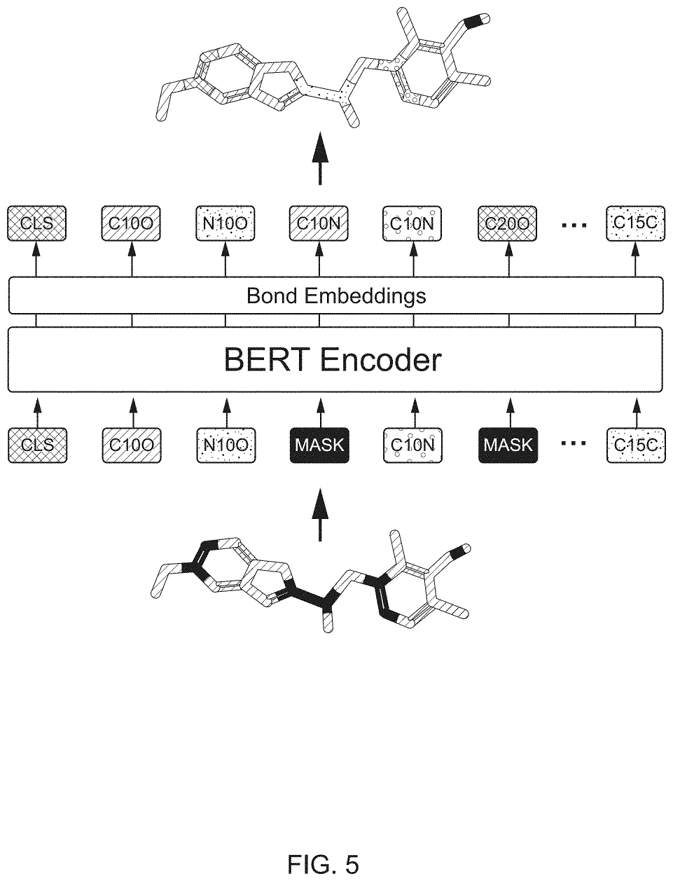

FIG. 5 depicts an overall process of training the masked bond model. Some of the tokens from the bond string representation of a molecule can be selected (e.g., randomly selected) and replaced with a [MASK] token. The corresponding bond positions of all mask tokens can be kept as is. The masked bond string and the bond position can be fed to the Encoder network of the Transformer model (e.g., a BERT encoder). The model loss is then computed using only the prediction at each masked position.

The masked input BSP representations and the predicted unmasked BSP representations for the example of FIG. 5 are:

### Input: C10O N10O [MASK] C10N [MASK] C10C C15C C15C C15C C10O [MASK] C15N C15C

### Output: C10O N10O C10N C10N C20O C10C C15C C15C C15C C10O C15N C15N C15C

The Transformer model can include the Encoder network and a Decoder network. Thus, pre-training the Transformer model can include training the Encoder network to learn how to transform the BSP molecular representation into an embedding space and training the Decoder network to learn how to transform a data point in the embedding space into the corresponding BSP molecular representation or into another representation that identifies the structure of a molecule, such as a Simplified Molecular-Input Line-Entry System (SMILES) representation.

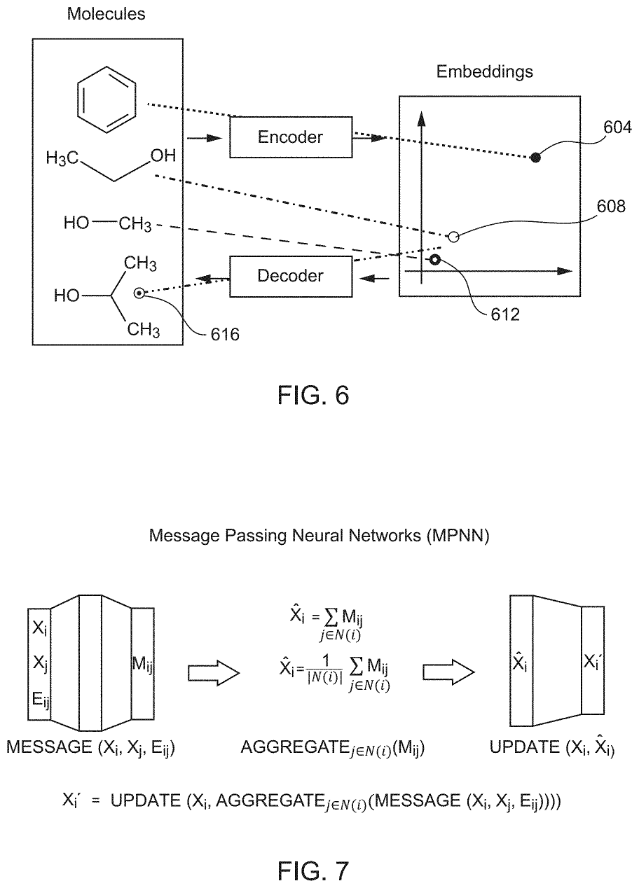

FIG. 6 shows an example of these transformations (while simplifying the number of dimensions in the embeddings space). In the illustrated instance, benzene's embedding (point 604) is far from the embeddings of methanol (point 608) and ethanol (point 612). The illustrated instance also shows (via point 616 and the line going to point 616) the Decoder transforming a given data point in the embedding space into a predicted molecule (corresponding to isopropyl alcohol).

II.E. Graph Neural Network for Generating Fingerprints of Molecules

Rather than using an Encoder network trained within a Transformer model to generate a fingerprint for a molecule, a Graph Neural Network (GNN) can be used to generate the fingerprint. A molecule can be interpreted as a molecular graph where atoms are nodes and bonds are edges. Under such representation, a GNN can be used to obtain the embedding of the molecule. A typical GNN may include multiple graph convolution layers. To update the node features, the graph convolution layer may aggregate features of the adjacent nodes. There are many variants of graph convolution. For example, a Message Passing layer may be particularly expressive and may allow incorporation of edge features that are important for molecular graphs.

FIG. 7 illustrates a representation of a Message Passing layer. Each node may collect messages from the neighboring nodes. Nodes are shown as Xᵢ and Xⱼ. A message may be sent from Xⱼ to Xᵢ, including edge information Eᵢⱼ. The message value is represented as Mᵢⱼ. The messages, Mᵢⱼ, may then be aggregated using a permutation invariant function such as mean or sum. The aggregated message is shown as \hat{X}_{ι}. Each node may update its feature vector using aggregated messages, \hat{X}_{ι}, and its own feature vector, Xᵢ. The updated node is shown as Xᵢ′.

II.F. GNN Pre-Training

Similar to Transformers, GNNs can be pre-trained on unlabeled molecules. Two methods of GNN pre-training include atom masking and context prediction.

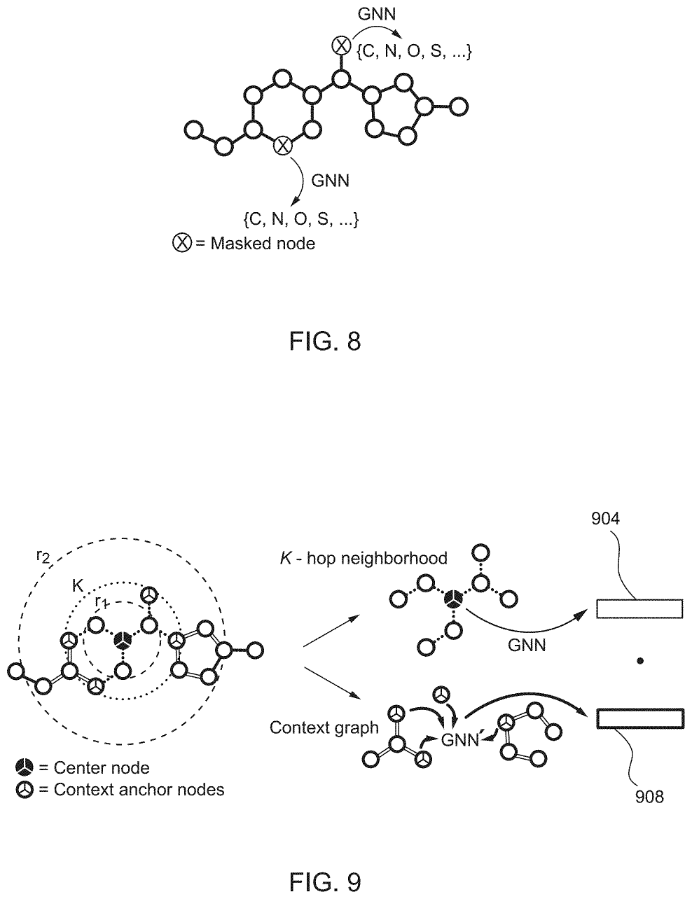

In atom masking, some nodes are selected (e.g., using a random or pseudo-random selection technique) and replaced with a MASK token. GNNs can then be applied to obtain the corresponding node embeddings. Finally, a linear model is applied on top of embeddings to predict a label of the masked node. FIG. 8 shows an approach for atom-masking pre-training. The “X” shows a masked node in a molecular graph. A GNN is used to obtain the identity of the masked node.

In context prediction, for each node v, neighborhood and context graphs of v may be defined as follows. K-hop neighborhood of v contains all nodes and edges that are at most K-hops away from v in the graph. This is motivated by the fact that a K-layer GNN aggregates information across the K-th order neighborhood of v, and thus node embedding h (K) v depends on nodes that are at most K-hops away from v. A context graph of node v represents the graph structure that surrounds v's neighborhood. The context graph may be described by two hyperparameters, r1 and r2, and the context graph may represent a subgraph that is between r1-hops and r2-hops away from v (i.e., it is a ring of width r2−r1). A constraint of r1<K can be implemented so that some nodes are shared between the neighborhood and the context graph, and those nodes can be referred to as context anchor nodes. Constraints may include K being 2, 3, 4, 5, 6, 7, 8, 9, or 10. These anchor nodes provide information about how the neighborhood, and context graphs may be connected with each other.

FIG. 9 shows molecular graphs and subgraphs for a K-hop neighborhood around a center node. K is the number of hops allowed between a given node and its neighbors, such that two nodes are considered neighbors if they are connected by a path with K or fewer edges. It is set to 2 in the figure. The context is defined as the surrounding graph structure that is between r1 and r2 hop from the center node, where r1=1 and r2=4 were used in the figure. FIG. 9 shows a subgraph for a K-hop neighborhood, including nodes within 2 hops of the center node. FIG. 9 also shows the context subgraphs created by extracting the portions of the molecule that fall between r1 and r2. Box 904 represents the embedding vector of the K-hop neighborhood subgraph, and box 908 represents the embedding vector of the context graph. The dot represents the vector dot product similarity.

Table 1 shows performance metrics of a GNN generated using different data sets, evaluation metrics and types of pre-training. Generally, performance metrics associated with the atom-mask pre-training slightly exceeded those associated with the context-prediction pre-training.

TABLE 1
Validation scores on DeepChem tasks

| | Number | Number | | Context | |
|---|---|---|---|---|---|
| Dataset | of | of | Evaluation | Prediction | Atom Mask |
| Name | Tasks | Samples | Metric | Pre-training | Pre-training |
| HIV | 1 | 41913 | ROC-AUC | 0.7633 | 0.7801 |
| BACE | 1 | 1522 | ROC-AUC | 0.8466 | 0.8630 |
| BBBP | 1 | 2053 | ROC-AUC | 0.7075 | 0.7312 |
| Tox21 | 12 | 8014 | ROC-AUC | 0.7473 | 0.7507 |
| ToxCast | 617 | 8615 | ROC-AUC | 0.6000 | 0.6300 |
| SIDER | 27 | 1427 | ROC-AUC | 0.6457 | 0.6629 |
| ClinTox | 2 | 1491 | ROC-AUC | 0.5000 | 0.5000 |

II.G. Directional Variational Transformer

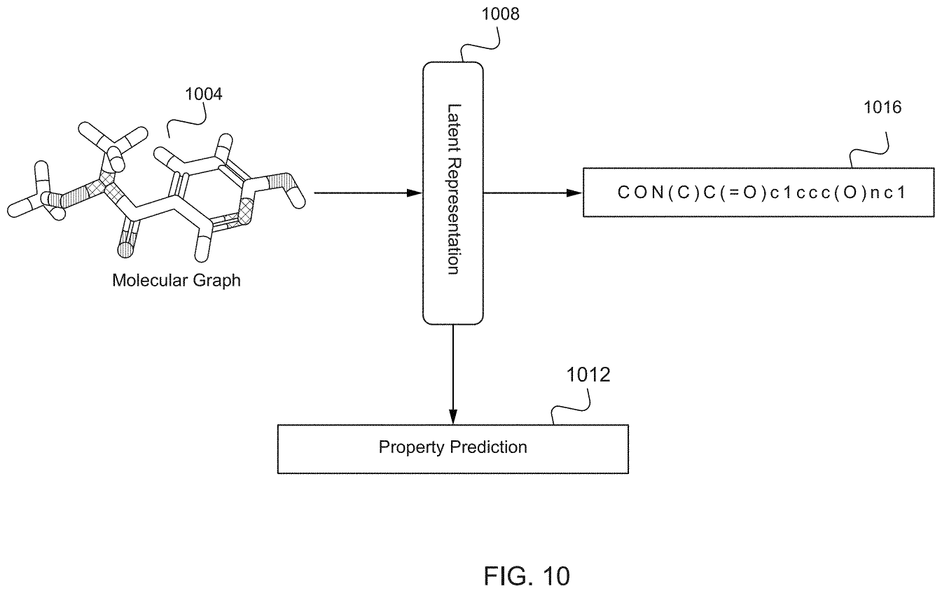

In order to predict properties of a molecule, the molecular graph may be represented as a fixed size latent vector such that the molecule can be reconstructed from the latent vector as a SMILES string. The size of the latent vector is a hyperparameter and may be determined empirically. This latent vector can then be used to predict the properties of the molecule. FIG. 10 illustrates molecular graph 1004 as an example. The molecular graph may be represented using BSP. Molecular graph 1004 can have latent representation 1008. Latent representation 1008 can be used for property prediction 1012. Additionally, a SMILES string 1016 can also be reconstructed from latent representation 1008.

One Encoder-Decoder architecture that may be used to generate a latent representation for property prediction is DVT (Directional Variational Transformer). With DVT, the Encoder network may be a graph-based module. Graph-based modules may include graph neural networks (GNNs). The graph-based modules may consider distance between atoms and the spatial direction from one atom to another. DimeNet (github.com/gasteigerjo/dimenet) is an example of a GNN that considers both atoms and spatial direction from one atom to another. Similar to DimeNet, the DVT may embed messages passed between atoms rather than the atoms themselves. A message passing layer is described with FIG. 7. The Decoder network may take the latent representation from the Encoder network as an input and then generate the SMILES representation.

Select differences between DVT and the variational transformers (VT) (e.g., model trained as variational autoencoder in section II.D.) include:
1. DVT model uses relative position based on angles and distance instead of absolute coordinates-based position.
2. Unlike VT, DVT does not perform global attention. Instead, DVT does attention with respect only to either bonds that share a common atom or neighboring bonds within a certain threshold distance. The threshold distance may include bonds within a maximum number of hops (e.g., every node at most two edges away) or a physical distance (e.g., 5 A).
3. DVT uses a separate readout node which aggregates the molecular graph to generate a fixed size latent vector.

The DVT model can be used in place of another transformer model. For example, the DVT model may be pre-trained and trained similar to the variational autoencoder model described in section II.D. Additionally, aspects of transformer models described with the DVT model may also apply to other transformer models.

II.G.1. Relative Position Generation

With respect to the Encoder network of the DVT, an input data set may represent a molecule in a manner that identifies a sequence of bonds with the position of its atoms in 3-D space. Using the positions of the atoms, the relative distances and angles between two bonds may be computed.

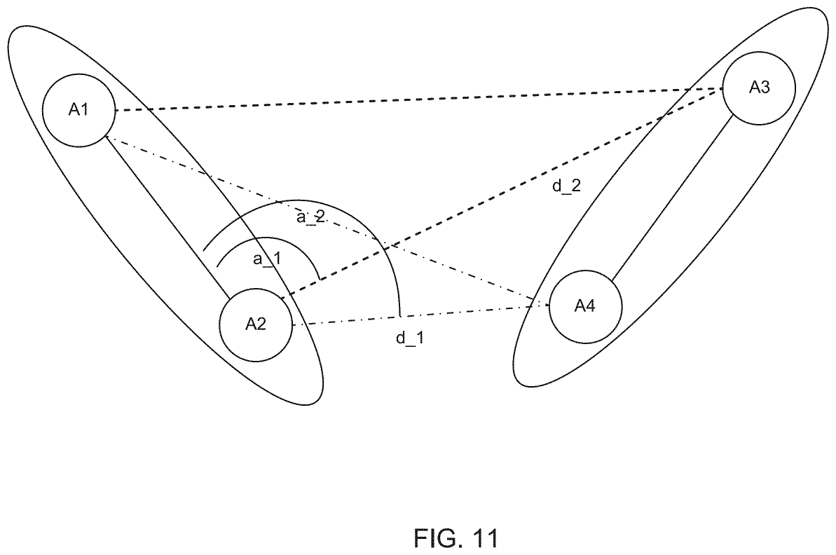

FIG. 11 illustrates the relative position in terms of angles and distances. FIG. 11 shows four atoms: A1, A2, A3, and A4. A1 and A2 are connected by a bond, and A3 and A4 are connected by a bond. B1(A1, A2) represents the bond between atom A1 and A2, and B2(A3, A4) represents the bond between atom A3 and A4. Distances between atoms and bonds are represented in the figure. The distance between two bonds is represented by [d1, d2] where dn=d(x, y) is the Euclidean distance between atoms x and y. For d1, x is A2 and y is A4. For d2, x is A2 and y is A3. The angle between two bonds is computed as [a1, a2] where a1=a(x, y, z) is the angle between line x-to-y and y-to-z. For example, for a1, x is A1, y is A2, and z is A3. For a2, x is A1, y is A2, and z is A4.

The order of the atoms in a bond may be defined by their order of appearance during a depth-first search over the molecular graph of the molecule. Because the molecular graph created from canonical SMILES is unique, the model can learn to generalize the order. Generalizing the order may refer to the model learning how to generate canonical ordering of SMILES during training, and when BSP of a molecule not seen during training is an input, the model can output the canonical SMILES of that molecule. Once the order of atoms in a bond is fixed, the second atom can be chosen to compute distances and angles. For example, FIG. 11 shows the distances and angles from atom A2 rather than atom A1, even though A1 and A2 are both in the same bond. Alternatively, the distances and angles can be computed from the first atom (e.g., A1) in the bond. The performance of the model may remain high regardless of whether the calculations of angles and distances are performed using the first atom of a bond or the second atom of a bond.

II.G.2. Encoder

A graph representation of a molecule may be input into the Encoder network. The graph representation may identify spatial relationships between atoms and/or spatial properties pertaining to bonds between atoms. In some embodiments, the graph representation may include a representation of the molecule in two dimensions. In some embodiments, the graph representation may include a representation of the molecule in three dimensions. The encoder network may produce a fixed size latent vector as an output. The Encoder network may include a plurality of heads. For example, the Encoder network may include two heads: a graph attention head and a readout attention head. Other heads that may be used include a standard attention head or an OutputBlock head, similar to what is used in DimeNet.

a. Graph Attention Head

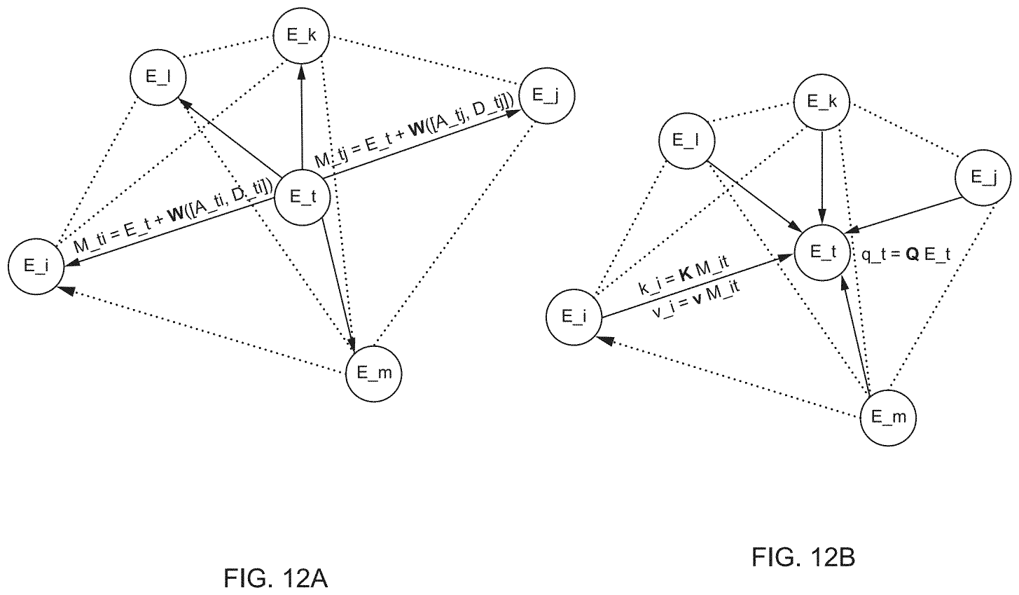

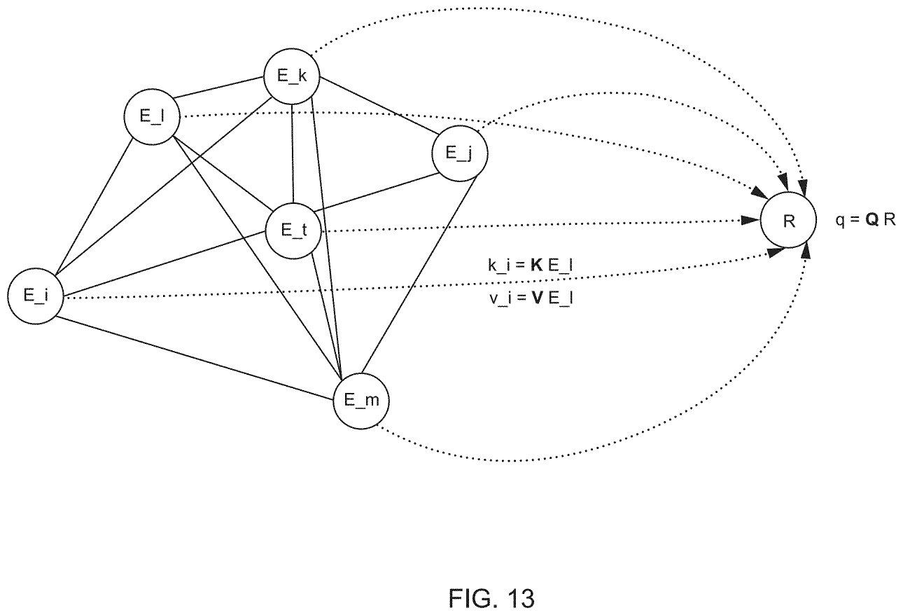

A graph attention head performs attention-based message passing between nodes to provide relative angle and distance information. As shown in FIGS. 12A and 12B, message passing may include two steps. In step 1, nodes may send messages to their neighboring nodes, a message from node i to node j is a combination of embedding of node i and a relative position of node i with respect to node j, as shown in FIG. 12A. The message includes an angle from E_t to another node (e.g., A_ti) and a distance from E_t to another node (e.g., D_ti). In some embodiments, all nodes may send messages to neighboring nodes. The number of message passing steps may be the same as the number of graph attention layers in the Encoder network. For example, the number of graph attention layers may be 2, 3, 4, 5, 6, 7, 8, 9, 10, or more. In step 2, each node may aggregate its incoming messages using an attention mechanism, as shown in FIG. 12B. Each node may generate a key-value pair (i.e., k and v) for all incoming messages. Each node also may generate a query vector (i.e., q) from its own embedding. Using the query and key, each node may compute the attention score by doing the vector dot product between query and key vector for all incoming messages. The updated embedding of the target node may be generated based on a weighted average of the value vector of all incoming edges. An example of the weight is W([A_ti, D_ti]), which is a vector that contains directional information between bond t and i in FIG. 12A. If attention is between bonds that share a common atom, then A_ti and D_tj are scalar values representing single angle and distance values. If attention is between bonds that are within a certain threshold distance, then A_ti and D_tj are vectors of size 2 and can be computed as shown in FIG. 11.

In this example, the attention scores are used as weights. For example, a set of embeddings may be generated, where each embedding represents an angle or distance of a bond in one or more molecules (e.g., such that the set of embeddings corresponds to a single ionic liquid molecule or a combination of a single ionic liquid molecule and a target molecule). For each particular embedding, a set of key-value-query pairs are generated that correspond to pairs between the particular embedding and each embedding in the set of embeddings. An attention mechanism (such as one described in Vasawni et al., “Attention is all you need,” 31^{st} Conference on Neural Information Processing Systems (2017)), which is hereby incorporated by reference in its entirety for all purposes) can be used to determine an extent to which to weight embeddings of various bond angles or bond distances when generating an updated embedding corresponding to a given bond angle or bond distance.

b. Readout Attention Head

A readout attention head may be another head used in the Encoder network. The readout head may aggregate all node embeddings to generate fixed size latent vectors using the attention mechanism. A readout node may be used to aggregate all nodes. The readout node may be a single node connected to all other nodes but excluded from the message passing mechanism. The readout node is R in FIG. 13. The readout node may act as a query vector and may have an embedding that is learned during training. Key-value pairs (i.e., k and v) from all nodes in the graph may be generated. Using query and key, the attention score may be computed for all nodes of the graph. Weighted aggregation of all value vectors where attention scores are weights may be performed.

II.G.3. Decoder

The fixed size latent vectors generated by the Encoder network may be an input to the Decoder network. The Decoder network may produce SMILES representation of the molecule as an output. The Decoder network may be trained to learn to transform a data point in the embedding space into SMILES representation.

### III. Fine-Tuned Network for Molecular Property Prediction

Because the Transformer model includes an Encoder network, pre-training the Transformer model may result in the Encoder network being pre-trained (so as to be able to transform BSP representations of molecules into representations in an embedding space).

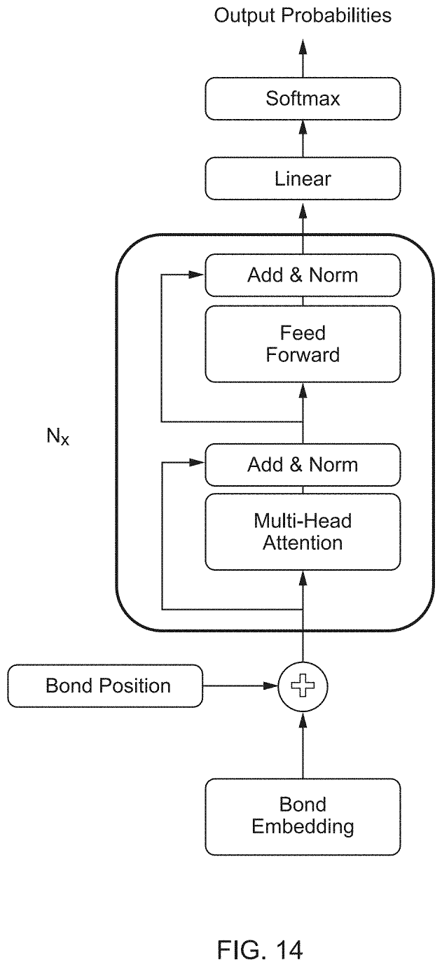

The Encoder network can then be fine-tuned such that BSP representations may be transformed into predicted values of a specific property (e.g., viscosities, densities, solubilities in a given solvent, activity coefficients or enthalpies) of a sample of the molecules. For example, a classifier or regressor can be attached to the output of the Encoder network, and the classifier or regressor can be fine-tuned on the specific task. FIG. 14 illustrates an exemplary model that includes the Encoder network that includes a linear layer with an activation function (e.g., Softmax function). This linear layer can be replaced with a classifier or regressor, and it can be fine-tuned to transform embedded values generated by the Encoder network to predicted property values.

The model shown in FIG. 14 further includes a multi-head attention layer. The multi-head attention layer may use query, key, value matrices to determine an extent to which to “attend” to each of a set of elements in a received data set (e.g., a BSP representation of a molecule) when processing another of the set of elements. More specifically, each element may be associated with three matrices: a query, a key, and a value. For a given element, the query may be defined to capture what types of keys to attend. Thus, the query can be compared to the same element's key and each other element's key (via a dot product) to generate a weight for the pair of elements. These weights can be used to generate a weighted average of the elements' values. The multi-head attention layer can perform this process multiple times using different “heads”. Each head can attend to different types of data (e.g., data from nearby elements, rare values, etc.). The model shown in FIG. 14 further includes an “add & norm” layer. This residual connection performs element-wise addition and layer normalization (across the feature dimension).

### IV. Reaction Prediction

The fine-tuned Encoder network (that includes the Encoder network and a regressor, classifier, and/or activation function) can generate property predictions of individual molecules, but the Encoder network does not offer a principled method of treating mixtures of molecules, such as ionic liquids composed of distinct cations and anions. Accordingly, a separate reaction prediction model can be generated that uses the same BSP representation described above but a different encoder/decoder model architecture.

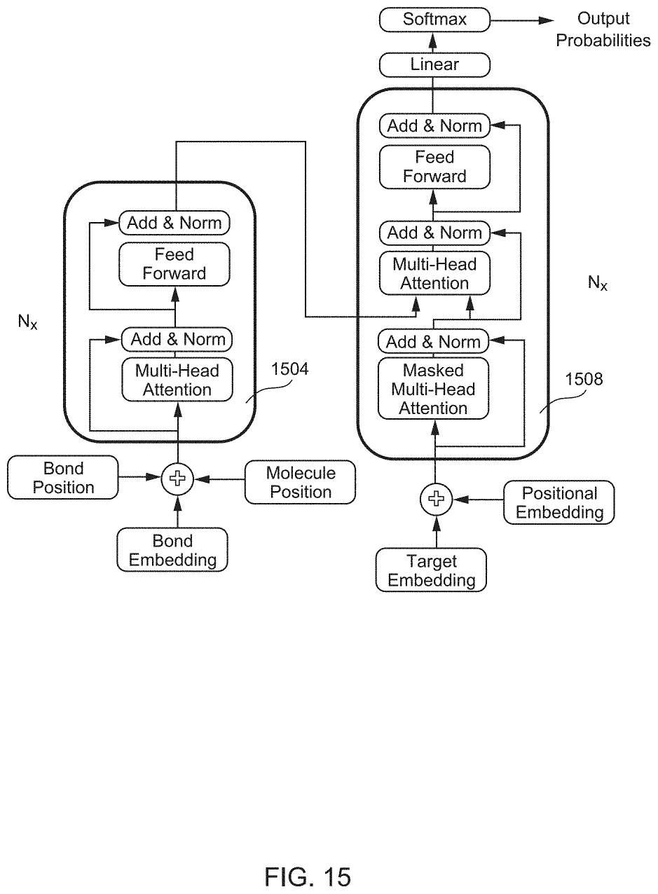

An exemplary architecture of a reaction prediction model is shown in FIG. 15. The model includes Encoder network 1504 and Decoder network 1508. Bond position, bond embedding, and molecule position are inputs into Encoder network 1504. The output of Encoder network 1504 is an input to Decoder network 1508. Embeddings of two molecules are also inputs into Decoder network 1508. The output of the model in FIG. 15 are probabilities for outputs of a chemical reaction.

Reaction prediction can be treated as a machine translation problem between BSPs of reactants and reagents and a representation of the products (e.g., a SMILES representation, SELFIES representation, or BSP representation). A SMILES representation for the product may be advantageous over the BSP representation since translating the latter into a human-readable chemical formula is nontrivial. A SMILES representation for the product may further be advantageous over the SELFIES representation because it may be more difficult for the model to infer the size of rings and branches when using the SELFIES representation.

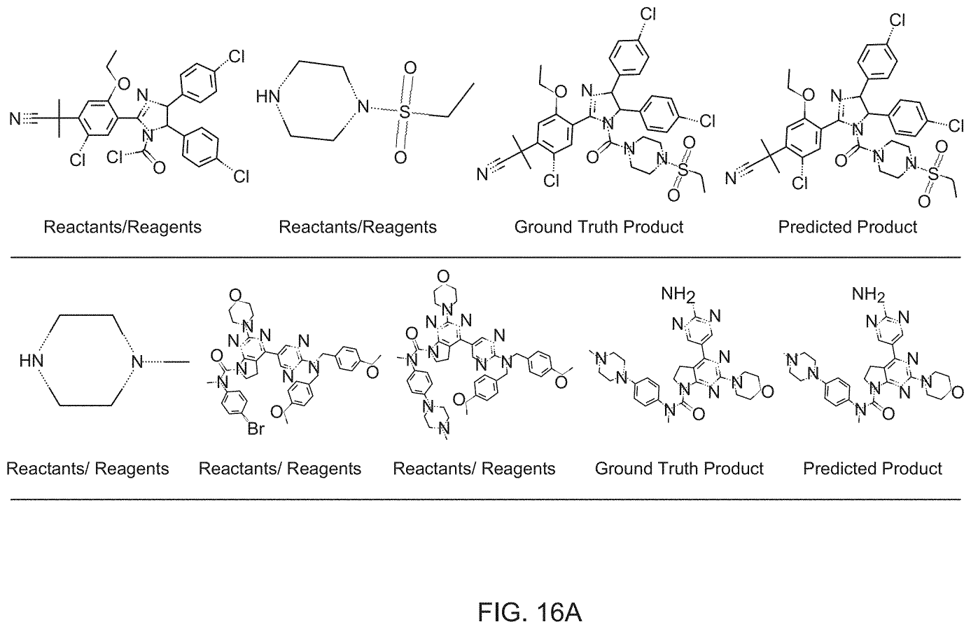

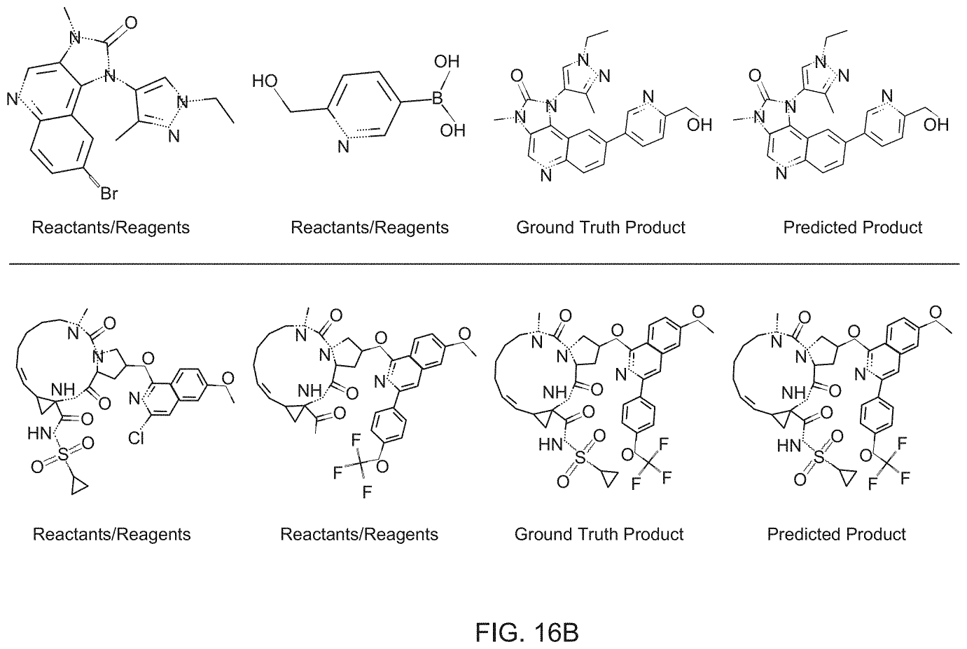

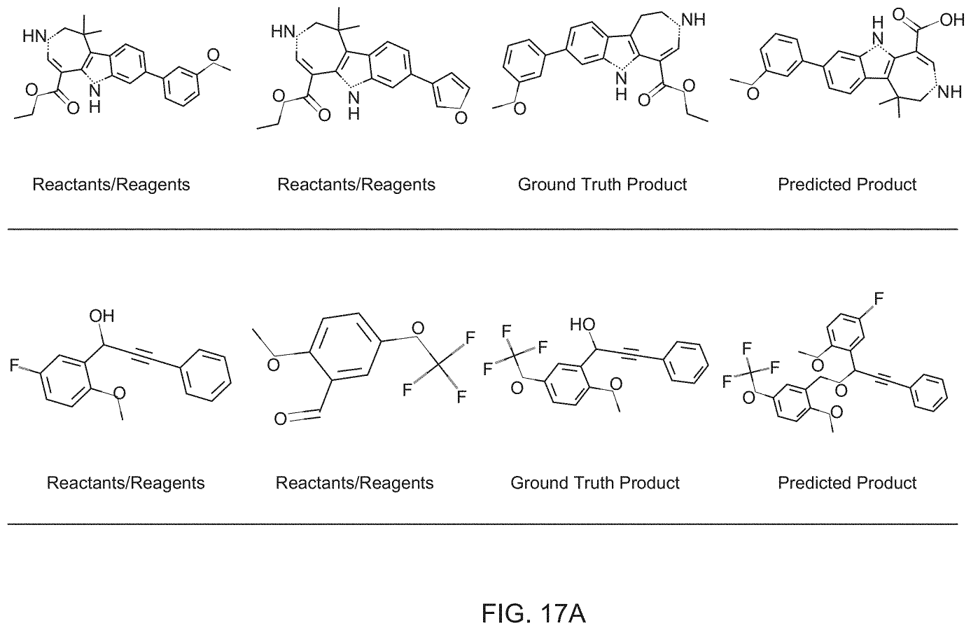

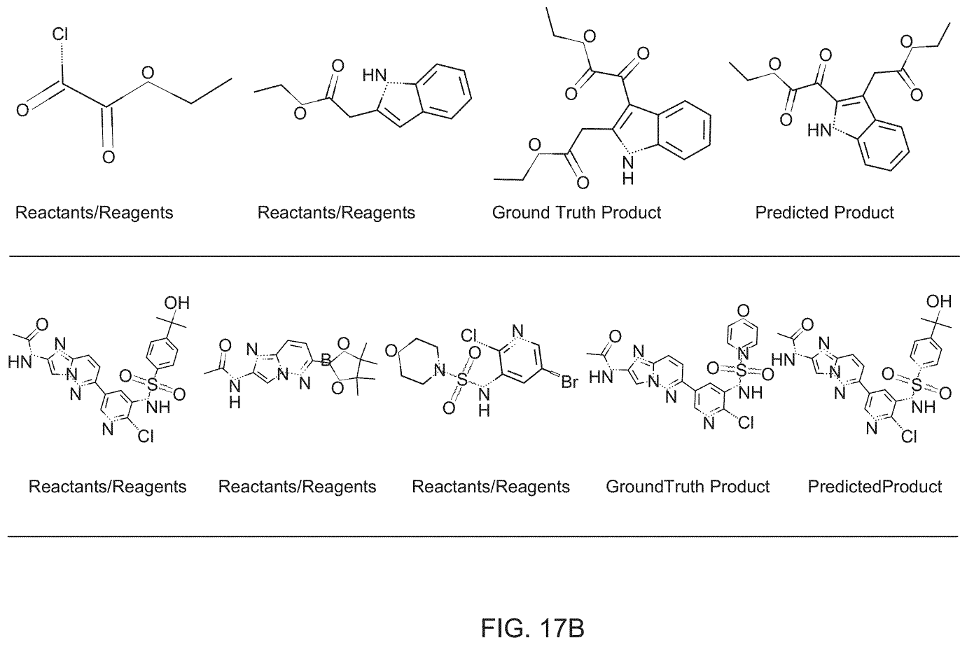

The training task for the separate reaction prediction model can be to learn to predict the products from reactants. FIGS. 16A and 16B show some of the success cases of the model. Each row includes the reactants and reagents and then the ground truth product formed and the predicted product. The predicted product matches the ground truth product. FIGS. 17A and 17B show some of the failure cases of the model. In FIGS. 17A and 17B, the predicted product does not match the ground truth product. One of the observed failure modes of the model is that instead of performing a reaction it copies one of the molecules from the reactants or reagents. This suggests that a failure mode is predicting no reaction from the reactants and reagents.

IV.A. Exemplary Results of a Reaction-Prediction Model

IV.A.1. DeepChem Tasks

To evaluate the BSP representation for molecular property prediction, a BERT masked bond model was trained on the molecules from MIT_USPTO and STEREO_USPTO datasets. The STEREO_USPTO dataset includes stereochemistry (i.e., orientation of bonds in a molecule) in its SMILES description, which makes the overall encoding/decoding more challenging. With STEREO_USPTO, the model predicts both the graph structure and the bond orientation. The BSP input to the model was constructed as described in FIG. 2. Since there is only one molecule in each sample, molecule position was not used. A classifier or regressor was then attached on top of the pre-trained BERT model and was fine-tuned on the downstream classification or regression tasks. The results on 10 different datasets from the DeepChem evaluation suite are shown in Table 2. The different tasks represent different properties of the molecules. For example, in the Tox21 dataset, the 12 tasks means that each molecule has 12 properties for the model to predict. Validation scores for USPTO datasets are shown in Table 3.

TABLE 2
Validation score on DeepChem tasks

| | Number | Number | | |
|---|---|---|---|---|
| Dataset | of | of | Evaluation | |
| Name | Tasks | Samples | Metric | Model score |
| ESOL | 1 | 1128 | RMSE | 0.45 |
| FreeSolv | 1 | 643 | RMSE | 0.97 |
| Lipophilicity | 1 | 4200 | RMSE | 0.61 |
| HIV | 1 | 41913 | ROC-AUC | 0.82 |
| BACE | 1 | 1522 | ROC-AUC | 0.73 |
| BBBP | 1 | 2053 | ROC-AUC | 0.96 |
| Tox21 | 12 | 8014 | ROC-AUC | 0.84 |
| ToxCast | 617 | 8615 | ROC-AUC | 0.82 |
| SIDER | 27 | 1427 | ROC-AUC | 0.84 |
| ClinTox | 2 | 1491 | ROC-AUC | 0.96 |

TABLE 3
Validation score on USPTO datasets measuring the accuracy
of the model's product predictions on the validation set.

| Dataset | Number of Samples | Top-1 validation accuracy |
|---|---|---|
| MIT_USPTO | 409035 | 85.65% |
| STEREO_USPTO | 902581 | 65.75% |

Table 2 shows that different datasets and tasks can result in a high RMSE or AUC evaluation metric. Table 2 shows the results for predicting molecular properties. Table 3 shows that the validation accuracy with the MIT_USPTO dataset is about 86% and STEREO_USPTO is about 66%. Validation scores for USPTO datasets are for predicting the product of the reaction.

IV.A.2. Ionic Liquid Property Prediction

A pure binary ionic liquid (IL) is made up of two molecules—namely a cation and an anion. Mixture ionic liquid can have more than one component. Since the masked language model is trained on single molecules, it is not useful to obtain embedding of ionic liquids. Therefore, the Encoder network of the reaction prediction model may be used to obtain an embedding of ionic liquids. To differentiate between pure binary ILs and mixture ILs, component number was used as an additional input after obtaining the embeddings. Table 4 shows exemplary validation scores for two properties (density and viscosity). The number of components refers to the number of ionic liquids. For example, if the number of components is 2, then there are 2 ionic liquids, which includes 2 cations and 2 anions.

TABLE 4
Validation score on ILThermo dataset

| | | Number of | Evaluation | |
|---|---|---|---|---|
| Dataset Name | Property Name | Components | Metric | Model score |
| ILThermo | Density (g/m³) | 1 | MAE | 10 |
| ILThermo | Density (g/m³) | 2 | MAE | 16 |
| ILThermo | Viscosity | 1 | MAE | 0.30 |
| | (log(Pa × s)) | | | |
| ILThermo | Viscosity | 2 | MAE | 0.34 |
| | (log(Pa × s)) | | | |

IV.A.3. Analysis of Attention Weights

To understand the model's ability to relate different bonds in three-dimensional space, attention weights of the reaction prediction model trained on the MIT_USPTO dataset were visualized. There are three types of attention i.e., self-attention on reactants/reagents, self-attention on product and attention between reactant/reagent and product.

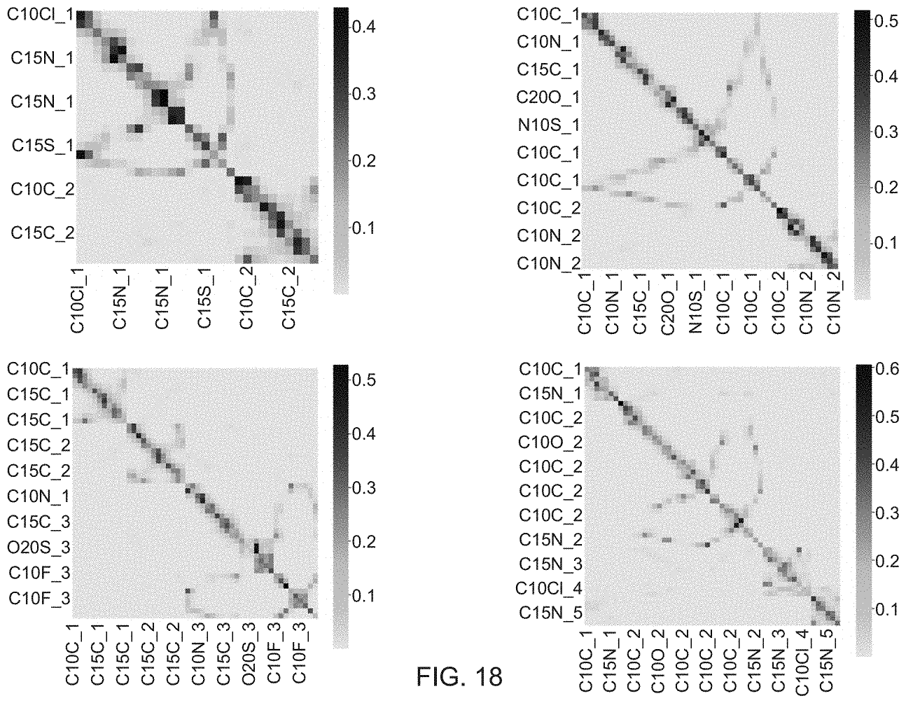

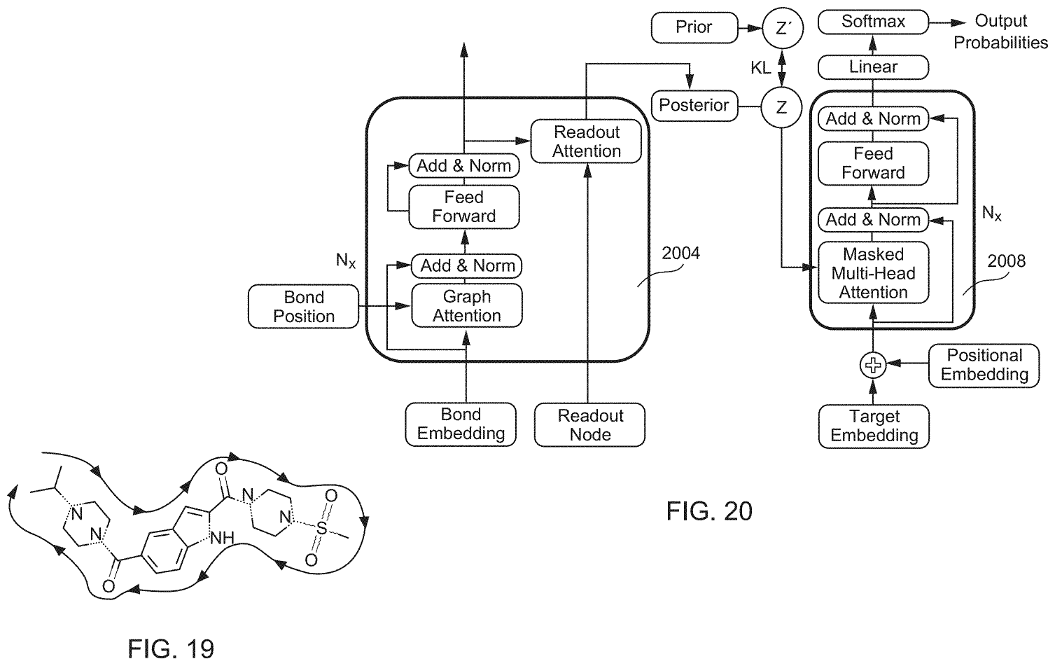

Reactant/reagent self-attention weights were extracted from the Encoder module of the model which takes BSP representation as an input. FIG. 18 shows the attention weights of the encoder's fourth head in its third layer. As shown, the model learned to find the neighboring bonds in three-dimensional space using the bond position which is evident from the diagonal nature of the attention maps. The bond string was generated by traversing the molecular graph as shown in FIG. 19. Thus, some of the neighboring bonds appear far apart in the bond string sequence which resulted in the wings-like pattern seen in the attention maps.

IV.B. Exemplary Results with Direction Variational Transformer

FIG. 20 illustrates exemplary architecture of a reaction prediction model using a DVT. The main component is the graph-based Encoder network 2004. A molecular graph may be inputted into Encoder network 2004. Encoder network 2004 may then output a fixed size latent vector. Encoder network 2004 may use the relative position based on angles and distance instead of absolute coordinates-based position. Decoder network 2008 may be the decoder network from the Transformer model (e.g., as shown in FIG. 15). The fixed size latent vector may be inputted into Decoder network 2008. Decoder network 2008 may then generate the SMILES representation of the molecule. As shown in FIG. 20 and not FIG. 15, Encoder network 2004 is configured to receive identifications of angles and distances as an input. Additionally, Encoder network 2004 (shown in FIG. 20) is not configured to receive bond position or molecule position (e.g., coordinates in BSP) as an input. However, in some embodiments, a molecular graph may be interpreted as a type of BSP representation, where the nodes of the graph are bond string tokens. The bond position vector (coordinates) can be converted into angles and distance values, which are then passed to graph attention head.

Table 5 shows performance data for the variational transformer (VT) and the directional variational transformer (DVT) models. DVT had a higher smoothness test result on 100,000 samples than VT. The smoothness test shows the percentage of randomly generated latent embeddings that when decoded using the decoder model gives valid molecules. A higher smoothness test value indicates better and smooth latent space. VT had a higher reconstruction accuracy than DVT. The reconstruction accuracy is the percentage of molecules from validation dataset that were reconstructed perfectly by the model. Higher reconstruction accuracy indicates better capability of the encoder/decoder model. DVT took fewer average iterations to find the target ionic liquid than VT. In this experiment, latent embeddings are obtained for all the ILs, and then discrete Bayesian optimization is performed to find how many iterations it takes to find IL with the lowest viscosity score. Lower number of iterations is more desirable. DVT required fewer dimensions to compress ionic liquids. In this experiment, latent embeddings are obtained for all ILs, and then the embedding vectors are compressed into fewer dimensions using PCA such that 99% variance is preserved. DVT can represent a molecule in lower dimensional space than VT, which allows for higher computing efficiency. Table 5 shows that DVT may be advantageous over VT in the number of iterations to find a target ionic liquid and the number of dimensions required to compress ionic liquids.

TABLE 5
Results comparing variational transformers

| | | | Average | Number of |
|---|---|---|---|---|
| | Smoothness | Recon- | iterations | Dimensions |
| | Test (100,000 | struction | took to find | Required to |
| Model Name | samples) | Accuracy | target IL | Compress ILs |
| Variational | 97.68 | 80.27 | 37.4 | 40 |
| Transformer | | | | |
| Directional | 98.47 | 77.04 | 23.3 | 37 |
| Variational | | | | |
| Transformer | | | | |

### V. Search for Candidate Molecules Using Computational Simulations and Variational Autoencoder

Materials/compound selection is a persistent and long-standing issue in many areas of materials science/chemical synthesis that is primarily time- and resource-constrained, given the lack of widespread use of high-throughput experimentation (HTE). One exemplary type of material selection is to identify an ionic liquid that efficiently and effectively depolymerizes a specific polymer (e.g., to facilitate recycling).

Some embodiments relate to using artificial intelligence and computing at least partly in lieu of and/or to supplement wet-lab approaches for selecting a material/compound fit for a given use case in a manner that requires a relatively small amount of time and/or a relatively small amount of resources. While evaluating millions of ionic liquid options experimentally would take years or perhaps decades, AI- and computation-powered models offer the ability to do so in practical timescales.

In some embodiments, a database may be generated that includes predicted liquid-viscosity and solubility properties for each of a set of ionic liquids. The solubility properties can include mixing enthalpy and an activity coefficient. The solubility properties may pertain to a given ionic liquid and a specific compound (e.g., a specific polymer). These predicted properties may be generated by executing simulations, including COSMO-RS (COnductor-like Screening MOdel for Real Solvents) simulations, which are based on quantum/thermodynamics methods. These simulations may allow for screening and/or filtering one or more existing ionic liquid libraries for particular IL and IL/polymer solution properties. Calculations of molecular dynamics and density functional theory (DFT) may also be performed. The COSMO-RS simulations can post-process quantum mechanical calculations to determine the chemical potential of each species in a solution and, from there, the other thermodynamic characteristics (e.g., mixing enthalpy and/or an activity coefficient). Quantum-mechanical and/or thermodynamical methods may include COSMO-RS or DFT.

While it may be quicker to run COSMO-RS simulations than to perform wet-lab experiments, COSMO-RS simulations may use a screening charge density as input. This charge density is acquired from computationally time-consuming density functional theory (DFT) calculations so this may make COSMO-RS simulations slow for compounds that do not have precomputed DFT results.

Running COSMO-RS simulations can include:
Retrieving the ionic liquid pairs (cation and anion) from a database and enumerating ionic liquids combinatorially by evaluating all possible cation-anion combinations. The database can include the COSMO-RS ionic liquid database ADFCRS-IL-2014, which includes 80 cations and 56 anions with precomputed density functional theory (DFT) results (corresponding to 4480 distinct ionic liquids) and/or COSMObase, which includes 421 cations and 109 anions with precomputed DFT results (corresponding to 45889 unique ionic liquids).
Identifying the solute (polymer). The solute may be associated with stored precomputed DFT results for the central monomer of a trimer and potentially also for the central few monomers of an oligomer for a few polymers.
Setting up COSMO-RS computations using specific mole fractions (e.g., 0.2 for the polymer, 0.4 for the ionic liquid cation, and 0.4 for the ionic liquid anion—or 0.8 for the ionic liquid if cation and anion are used in conjunction) to replicate an example experimental setup for depolymerization using ionic liquids. Temperature and pressure can be set or retained to the values used by default in COSMO-RS calculations.
Obtaining solution mixing enthalpy and activity coefficient of the polymer in ionic liquid solution from COSMO-RS output and storing the results in a database.

The predictions can be used to perform a simple screening of IL cation and anion pairs to select an incomplete subset of ionic liquids that have the lowest predicted solution mixing enthalpies and activity coefficients. It can be verified that viscosity of the incomplete subset of ionic liquid at the depolymerization temperature and pressure is reasonable for mass transfer (e.g., from the ILThermo database directly or from the predictive model-based on transformer embeddings). The subset can be further filtered based on the depolymerization temperatures and pressures that can be identified.

Thus, in some embodiments, these simulations may be performed for only an incomplete subset of a universe of ionic liquids and one or more property-prediction models can be used to predict solubility properties for the other ionic liquids. Each of the property-prediction model(s) can be trained and/or fit using representations of the ionic liquids in the incomplete subset and corresponding values for a given property. For example, a first model may be defined to predict mixing enthalpies (corresponding to the specific compound [e.g., the specific polymer]), a second model may be defined to predict activity coefficients (corresponding to the specific compound), and a third model may be defined to predict IL viscosities. The representations of the ionic liquids may include BSP representations of the ionic liquids. A model may be trained on the embedding space and relate embeddings of molecules to property values. Each of the property-prediction model(s) can include a regression model.

One or more generative models can be used to interpolate between representations of candidate ionic liquids in order to explore the embedding space (into which initial molecule representations are transformed using, for example, an Encoder network) and discover desired enhanced IL and IL/polymer solution characteristics for polymer degradation reactions. That is, the generative model can explore additional molecules and can map the embeddings of these molecules to values of one or more properties. The generative model(s) may include (for example) a regression model. Using the generative model, continuous predictions can be generated across the embedding space.

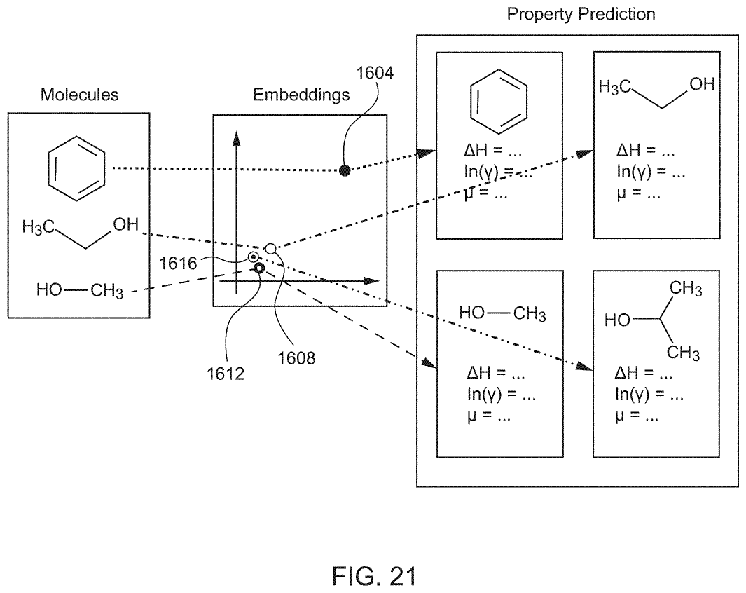

For example, FIG. 21 illustrates how individual points in the embedding space can correspond to a given chemical structure (that may be predicted using a reconstruction Decoder) and to predicted properties. In embodiments, the generative model may refer to a model that generates an output, identifying molecules (e.g., ionic liquids) that are not yet known to be useful for a particular context (e.g., depolymerization). The predicted properties may be identified using another model (e.g., a regression model on top of a generative model, that translates a given position in the embedding space into a predicted property).

In some instances, a single model (e.g., a single generative model with a regression task) can be configured to generate multiple outputs for any given position within an embedding space, where the multiple outputs correspond to multiple properties. For example, the single model may generate a predicted mixing enthalpy, activity coefficient and viscosity for a given position in the embedding space. In some instances, multiple models are used, where each model generates predictions corresponding to a single property. For example, a first model may predict a mixing enthalpy, a second model may predict a viscosity, and a third model may predict an activity coefficient.

In the embedding-space representation shown in FIG. 21, data points 2104, 2108, and 2112 are shown. These data points are associated with measured values of properties, and data point 2116 is associated with no measured property value. In the depicted example, the benzene molecule (represented by data point 2104) is predicted to have unfavorable properties, while the methanol molecule (represented by data point 2112) and ethanol molecule (represented by data point 2108) are predicted to have favorable properties for the specific polymer degradation reaction. After a regression model was used to predict properties in the embedding space, a representation of a promising molecule (represented by data point 2116) is identified. Data point 2116 is close in the embedding space to data points 2108 and 2112. Given that molecules that are represented by data points 2108 and 2112 have favorable properties, the proximity of data point 2116 suggests that the molecule represented by data point 2116 will also have favorable properties. The “promising molecule” can correspond to a position in the embedding space where a condition associated with each predicted property is satisfied.

For example, a promising molecule can be associated with a predicted viscosity below or above a viscosity threshold; a predicted activity coefficient of a polymer in a solution of the promising molecules is below an activity-coefficient threshold; and a predicted mixing enthalpy for a solution of the promising molecules and the polymer being below a mixing-enthalpy threshold. That is, predictions generated by the generative model(s) can be used to identify positions within the embedding space that correspond to desired properties of interest (e.g., predicted viscosities that are below/above a predefined viscosity threshold, mixing enthalpies that are below a predefined mixing-enthalpy threshold, and/or activity coefficients that are below a predefined activity-coefficient threshold).

As another example, a score may be generated for each of some or all of the ionic liquids represented by a position within the embedding space. The score may be defined to be a weighted average that is based on predicted properties of interest. The values of the predicted properties of interest may be normalized. A higher score may indicate a more promising molecule. To illustrate, the score may be defined to be a weighted average of the predicted normalized viscosity (including log viscosity) or the negative of a predicted normalized viscosity (including log viscosity), the negative of a predicted normalized mixing enthalpy, and/or the negative or positive of a predicted normalized activity coefficient (including log activity coefficient). One or more promising ionic liquids may be identified as those corresponding to the n highest scores (e.g., as corresponding to the highest score or as corresponding to any of the highest 10 scores) or as those corresponding to a score above an absolute threshold. Alternatively, one or more promising ionic liquids may be identified as those corresponding to the n lowest scores (e.g., as corresponding to the lowest score or as corresponding to any of the lowest 10 scores) or as those corresponding to a score below an absolute threshold.

In some instances, a variational autoencoder is used to transform representations of data points (e.g., BSP representations) to predict the molecular structure and/or identity corresponding to the data points. The variational autoencoder may be configured with the Encoder network that transforms representations of molecules (e.g., BSP representations) into the embedding space distribution (e.g., mean and standard deviation of embedded representations) and a reconstruction Decoder network that is configured to transform representations sampled from the embedding space distribution back into the initial-space representations of molecules. In some instances, the reconstruction Decoder network is configured to generate SMILES representations of molecules instead of BSP representations (as the SMILES representations may be more interpretable to human users). In order to calculate the loss for a given prediction, an initial BSP representation may be transformed into a corresponding initial SMILES representation. A penalty can then scale based on a difference between initial SMILES representations and Decoder-predicted representations. Or the generated SMILES representation can be transformed into BSP representation and a penalty can then scale based on a difference between initial and final BSP representations.

Then the trained Decoder network from the variational autoencoder can be used to transform points of interest (e.g., associated with predictions satisfying one or more conditions) to the SMILES representations, such that the molecules can be identified. The points of interest may correspond to (for example) a local or absolute maximum of a given predicted value, a local or absolute minimum of a given predicted value, a given local or absolute maximum or a score that depends on multiple predicted values, etc. For example, a score may be defined to be negatively correlated with mixing-enthalpy predictions, activity-coefficient predictions, and viscosity predictions, and points of interest may correspond to the highest scores.

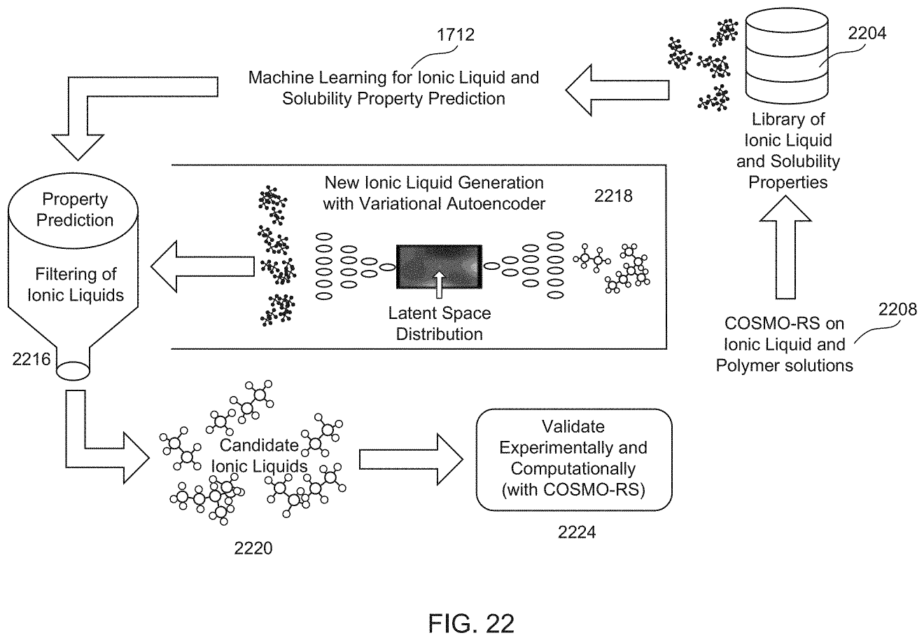

FIG. 22 illustrates an exemplary workflow for identifying candidate molecules for a reaction (e.g., depolymerization) using computational simulations and variational autoencoder. At stage 2204, a data store is created and/or accessed that includes one or more properties and structures of molecules. For example, the one or more properties can include viscosities, solubilities, enthalpies, and/or activity coefficients. To identify these values, COSMO-RS computations at stage 2208 may be accessed corresponding to compounds that have already precomputed quantum mechanical (DFT) calculations.

At stage 2212, one or more models (e.g., including generative and regression models) may be defined (using the above properties and representations of molecules, such as BSP representations) to relate the molecular representations to predicted molecular properties across a space. Each model may be specific to a polymer system to explore a space of various molecules corresponding to ionic liquids that may be used to depolymerize the polymer. Property values predicted by the model may influence whether a solution of molecules of a given type (associated with independent-variable positions in an embedding space) are predicted to depolymerize the polymer.

At stage 2216, the model(s) can thus generate, for each position within the embedding space, one or more predicted properties of a molecule (or a solution of the molecules) that correspond to the space.

Using the generative and regression models, one or more regions (e.g., one or more positions, one or more areas, and/or one or more volumes) within the embedding space that correspond to desirable properties can be identified. What constitutes the “desirable” properties may be defined based on input from a user and/or default settings. For example, a user may be able to adjust thresholds for each of one or more properties in an interface. The interface may update to indicate for how many molecules the threshold criteria are fulfilled.

At stage 2220, a Decoder network (trained within a corresponding variational autoencoder 2218) can then be used to transform data points within the one or more regions of the embedding space into structural representations of the molecules (e.g., SMILES or BSP representations of the molecules). Thus, a Decoder network of the variational autoencoder may be trained to reliably translate data points from the embedding space into a space that clearly conveys molecular structure. Given that the outputs from the generative and regression model(s) may identify positions within the embedding space(s) having properties of interest, one or more candidate molecules of interest can be identified using the variational autoencoder.

At stage 2224, properties of the one or more candidate molecules can then be experimentally measured. Such experiments may confirm a selection as a molecule of interest or may be used to update the embedding space, one or more transformations and/or one or more selection criteria.

V.A. Exemplary Results of Variational Autoencoder

A transformer-based variational autoencoder model was trained on a large data set of chemical molecules containing both charged (i.e., ionic liquid cations and anions) and uncharged species. The autoencoder model was then used to generate new ionic liquid cations by exploring the space near known ionic liquid cations.

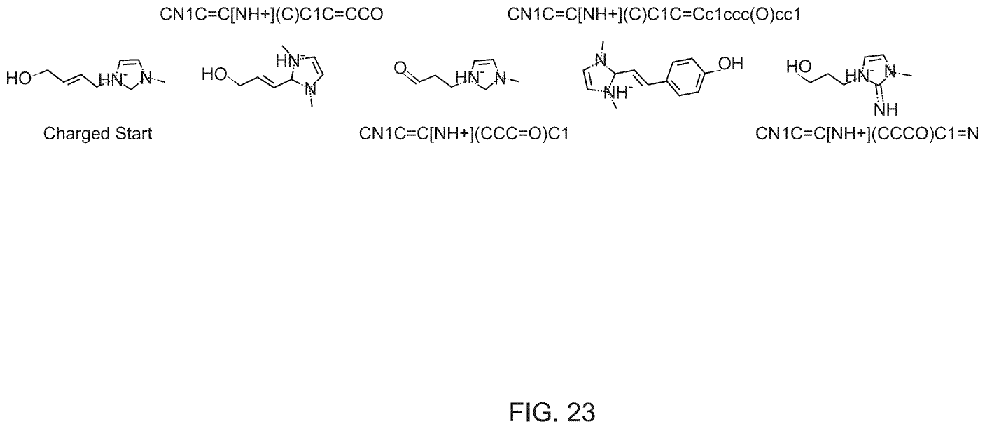

FIG. 23 illustrates exploring the space around a molecule to generate new ionic liquid cations. The starting ionic liquid cation is on the left and labeled “CHARGED START.” Random Gaussian noise was added to its embedding. The four ionic liquid cations were then decoded from the embedding. The SMILES string of each cation is listed underneath the four resulting ionic liquid cations. The four decoded outputs are all charged molecules, although this is not always the case because of the stochastic nature of the model. FIG. 23 demonstrates that additional ionic liquid cations can be generated from a single ionic liquid cation using a transformer-based variational autoencoder model.

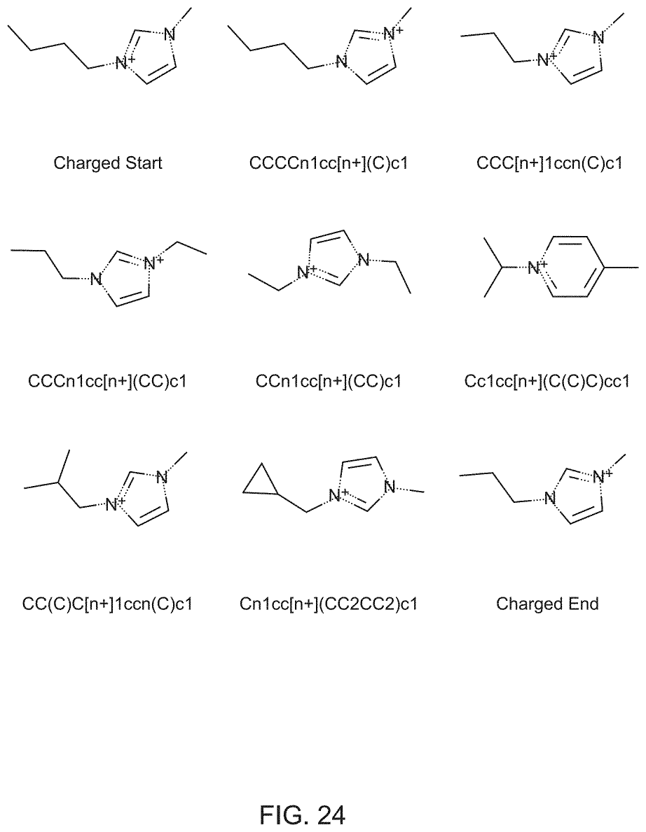

FIG. 24 shows exploring the space in between two molecules by means of linear interpolation. The two molecules are the ionic liquid cation on the top left labeled “CHARGED START” and the ionic liquid cation on the bottom right labeled “CHARGED END.” Again, because of the stochastic nature of the model, not all intermediate molecules need to correspond to charged species. FIG. 24 shows seven ionic liquid cations generated in the space between CHARGED START and CHARGED END. The SMILES strings are displayed underneath the generated cations. Other interpolation techniques, like SLERP (spherical linear interpolation), can also be utilized. FIG. 24 demonstrates that additional ionic liquid cations can be generated by exploring the space between two ionic liquid cations using a transformer-based variational autoencoder model.

Similar findings can be obtained for ionic liquid anions, and new ionic liquids can be generated by combining different generated cations and anions.

### VI. Ionic Liquid-Based Depolymerization Optimization

Chemical recycling involves reaction processes where waste (e.g., plastic waste) is converted back into molecular constituents to then be used as fuel or feedstock by the chemical industry. Optimized chemical reactions are selected by attempting to maximize conversion (amount of plastic dissolved relative to amount of starting plastic), yield (amount of monomer relative to amount of starting plastic), and selectivity (amount of monomer relative to total amount of reaction products). These deconstruction reactions are complex, as they depend on intricate coupling between chemical and physical processes spanning multiple time and length scales, meaning that design of experiments (DOE) is expensive and not easily generalizable across chemical space.

Chemical reaction optimization involves maximizing a function (e.g., a utility function) that is dependent on a set of reaction parameters. Although a variety of optimization algorithms have been developed (e.g., convex approaches for local minima such as gradient descent, conjugate gradient, BFGS, and non-convex, black box function optimization approaches for global optima such as systematic grid-searches), they can require many evaluations of the function and are not suitable for expensive processes such as laboratory chemical reactions where each evaluation requires a new benchtop experiment to be run.

An alternative approach for chemical reaction optimization uses the technique of Bayesian optimization via Gaussian Processes (GP). This technique can achieve good accuracy with limited evaluations. However, Bayesian optimization via Gaussian Processes is computationally expensive because it has poor scalability to high parameter dimension, and computationally scales as N³ where N is the number of training data points used to seed the Bayesian optimization.

Some embodiments disclosed herein include techniques for reducing dimensionality and requiring only a limited number of training (seed) data while performing Bayesian optimization via Gaussian Processes. The chemical and experimental space can then be efficiently navigated to identify an ionic liquid with favorable properties.

Application of Bayesian optimization to depolymerization reactions with ionic liquids is challenging. The number of ionic liquids that are possible may be large. Ionic liquids include a cation and an anion, and there may be thousands of possible cations and anions or hundreds of commercially accessible cations and anions. The interactions between the ionic liquids and the polymer to be depolymerized may not be well understood. The mechanism for how ionic liquids break down a polymer may not be known. Bayesian optimization therefore may be more difficult to apply to depolymerization than to other contexts. For example, depolymerization may be more complicated than pharmaceutical reactions where the desired drug (or a molecule that may have some properties desired of a drug) is expected to bind to some part of a target (e.g., pocket of a protein). As a result, Bayesian optimization of depolymerization may be more difficult to arrive at a converged solution than Bayesian optimization of pharmaceutical reactions.

VI.A. Dimensionality Reduction of Ionic Liquids

Bayesian optimization may be improved by using a reduced dimensionality space. The structure and/or other physical properties of an ionic liquid may be represented by a high dimensionality space (e.g., BSP representation or SMILES representation). A reduced dimensionality space that has similar molecules located close to each other may aid Bayesian optimization. A matrix can be used to represent the high-dimensional structure of ionic liquids. A dimensionality-reduction technique can then process matrices corresponding to various ionic liquids to identify a set of new dimensions. The set of new dimensions may capture at least 80%, 85%, 90%, 95%, or 99% of the variability of dependent variables with the higher dimensional space. Principal component analysis (PCA) is one possible dimensionality reduction technique. Given a collection of points in some high dimensional space, PCA finds the directions in that space along which the points vary the most. By selecting a small number of these directions and projecting the points along them, a lower-dimensional description of each point may be obtained. Within this space, molecules with fingerprint similarity (e.g., structural similarity and/or thermodynamic property similarity) are then located close to each other, while molecules that lack fingerprint similarity are located far from each other. This new space may include a relatively small number of dimensions (e.g., less than 30, less than 20, less than 15, less than 10, less than 8, or less than 5). This new space may include any embedding space described herein, including embedding spaces derived from BSP representations or GNNs (e.g., DVT).

As an example, a training set can be defined to include representations of binary ionic liquids with known viscosities. For each binary ionic liquid represented in the training set, cations and anions can be converted to SMILES, BSP, or other suitable representations. The representations can then be converted into descriptors having a fixed length, such as having fewer than 10,000 values, fewer than 7,500 values, fewer than 5,000 values, or fewer than 2,500 values. A single feature matrix representation of the ionic liquid can be generated by concatenating the anion and cation descriptors. The feature matrix may be normalized. The feature matrix can then be decorrelated by computing pairwise correlation between columns. If a correlation analysis detects a correlation between two columns (e.g., by detecting that a correlation coefficient is above an upper threshold or below a lower threshold), one of the columns can be removed from the feature matrix and separately stored. The dimensionality of the feature matrix may then be reduced to aid Bayesian optimization. A dimensionality-reduction technique (e.g., a component analysis, such as principal component analysis [PCA]) can be used to transform each feature matrix and other feature matrices into a set of components and respective weights (e.g., explained variance). A given number (e.g., a predefined number, such as 4, 5 to 10, 10 to 15, 15 to 20, or 20 to 30) of components associated with the highest weights (e.g., explained variance) can be used as a reduced-dimensionality representation of the ionic liquid.

In some embodiments, dimensionality of ionic liquids may be reduced by using embedding space described herein instead of PCA. The ionic liquids may be represented using mordred descriptors or with BSP-based embedding; descriptors that map similar molecules closer together. The dimensionality may be reduced using techniques such as PCA.

VI.B. Bayesian Optimization of Reaction Conditions Using Discrete Sampling

Within the reduced dimensionality space, Bayesian optimization via Gaussian Processes can be used to detect individual positions within the space for which experimentation data will be particularly informative in characterizing how a property (e.g., an output such as conversion or yield) varies throughout the space. For example, each position within the space may correspond to an ionic liquid, and a use case may include determining conversion and yield of a depolymerization reaction.

A function is constructed to estimate how an output varies throughout the reduced dimensionality space using an initial training data set and data determined by a Gaussian Process (GP). The initial training data set may include a measurement of a given property for each of multiple ionic liquids. Positions within the reduced dimensionality space that correspond to the multiple ionic liquids are identified, and a Gaussian Process prior is defined using the properties and positions (within the reduced dimensionality space) of the multiple ionic liquids. A posterior is then formed by computing the likelihood of an experimental outcome at each of one or more positions by updating the Gaussian Process prior with the positions and properties corresponding to the seed experiments. The posterior and a specified acquisition function can be used to construct a utility function, which is a concrete form of the acquisition function in which all optimization hyperparameters have been set. A position within the space that is associated with the highest value of the utility function can be selected, and an ionic liquid and other reaction parameters corresponding to the position can be identified for the next experiment (to collect a measurement for the given property of the ionic liquid and the reaction parameters). After the experimental data is collected, the posterior can be updated with the measurement, the utility function can be updated, and a different position can be identified for further experimental data collection.

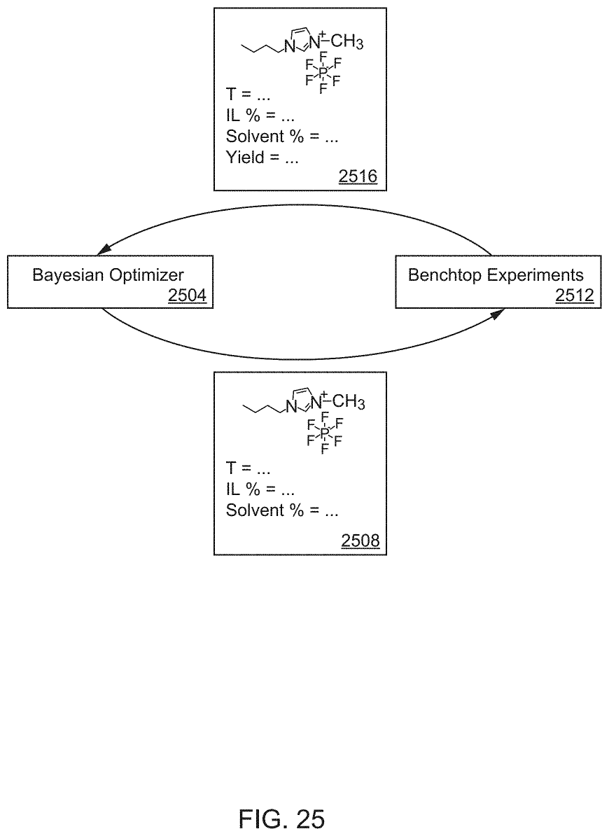

FIG. 25 illustrates the interaction between Bayesian optimization and benchtop experiments. An initial output of the Bayesian Optimizer 2504 may provide values for parameters shown in box 2508. These parameters may include the identities of the ionic liquids (e.g., cation and anion), the temperature T, the ionic liquid percentage, and the solvent percentage. A benchtop (or other scale) experiment 2512 may be performed. The benchtop experiment may provide a value for the yield (box 2516). This yield is associated with the identities of the ionic liquids, the temperature T, the ionic liquid percentage, and the solvent percentage. The yield is used to seed Bayesian Optimizer 2504 and the process is repeated until Bayesian Optimizer 2504 converges or an optimization constraint (e.g., number of iterations) is satisfied. The optimizer may navigate through the reduced dimensions in the learned embeddings created by the model generating multi-dimensional representations of molecules. The model may recommend entirely new molecules based on the embedding space. The variational autoencoder's embedding space may be used to seed the optimization routine.

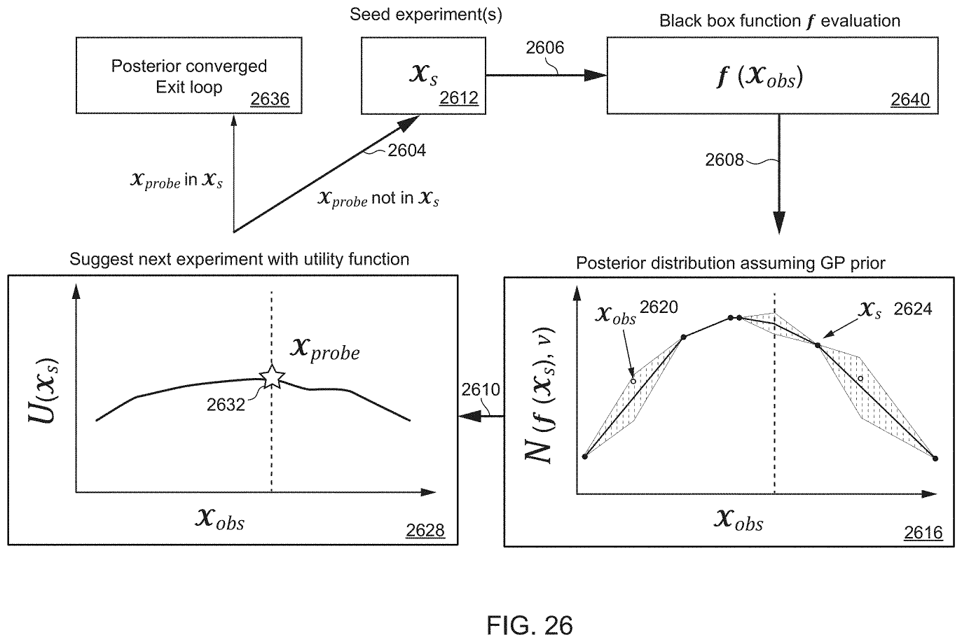

FIG. 26 illustrates a Bayesian optimization process via GP iterative loop using discrete sampling, x_{obs}. Thicker lines 2604, 2606, 2608, and 2610 show the loop which starts with a seed set of experimental results 2612 which are used to construct the posterior distribution 2616. Green points (e.g., point 2620) are x_{obs} and red points (e.g., point 2624) are all seed experiments (xₛ). The utility (shown in graph 2628), trained on all seed experiments, is calculated for all x_{obs}. The next experiment in the star 2632, x_{probe}, is picked from x_{obs}, because x_{probe} has the highest utility. If X_{probe} has been suggested before, the optimization has converged to a solution (box 2636) since the posterior distribution 2616 will not change. If X_{probe} has not been suggested before, the loop continues, following thicker lines 2604, 2606, 2608, and 2610. Discrete sampling may be applied in Bayesian optimization even when the value of the parameter may be continuous.

First, the Gaussian Process prior is constructed. The Gaussian Process (GP) is defined by the property that any finite set of N points {xₙ∈X}ₙ₌₁^{N} induces a multivariate Gaussian distribution on R^{N}. The surrogate function for black box function ƒ(x) (box 2640) assumes GP over R^{N}, but the prior is constructed using only a specific and finite set of points called x_{obs}. In other words, xₙ=x_{obs}, and x_{obs} is discrete. The set of points x_{obs} includes points that have been experimentally evaluated, providing experimental data. R^{N} describes the design space and may include parameters like solvent type, solvent concentration, solvent ratio, and reaction temperature.

Second, the posterior is constructed (e.g., posterior distribution 2616). Seed experiments are of the form {xₛ, yₛ}^{N}ₛ₌₁, where xₛ and yₛ are known experimental results for N number of cases (i.e., xₛ is a set of experimental conditions, and yₛ is the observed result). {xₛ, yₛ}^{N} can be thought of as the training set. Under the assumption that the black box function ƒ(x) is drawn from GP prior where yₛ˜Normal(ƒ(xₛ), v) and v is the variance of noise introduced by observable xₛ, the posterior is formed over functions. Seed experiments need to be supplied to construct the posterior. Seed experiments are a subset of the observables (xₛ ⊂x_{obs}).

Third, the next experiment is determined. The posterior along with a specified acquisition function is then used to construct the utility function, U(xₛ), which is a concrete form of the acquisition function in which all optimization hyperparameters have been set. In embodiments, the acquisition function may be the GP Upper Confidence Bound (UCB), which minimizes regret over the course of optimization:

a_{UCB}(xₛ;{x_{obs},y_{obs}},θ)=μ(xₛ;{x_{obs},y_{obs}},θ)+κσ(xₛ;{x_{obs},y_{obs}},θ).

κ corresponds to the degree of exploration versus exploitation (a low kappa indicates exploitation while a high kappa corresponds to exploration). Θ are hyperparameters for the GP regressor. The next experiment is proposed via a proxy optimization:

x_{probe}=argmaxₓU(x_{obs}).

Fourth, given X_{probe}, the black box function is evaluated, obtaining ƒ(x_{probe}). The black box function may be a simple lookup that maps x_{probe} to ƒ(x_{probe}), as in examples described herein. During testing, ƒ(x_{probe}) is obtained from running an actual experiment. The posterior may be updated given the result of ƒ(x_{probe}), as in the second step above. The third step of determining the next experiment may be repeated for the updated posterior. The fourth step of evaluating the black box function with the new ƒ(x_{probe}) may then be repeated. If x_{probe} is in the set of xₛ, then the Bayesian optimization loop may end because the optimization is considered to have converged.

VI.C. Example Methods

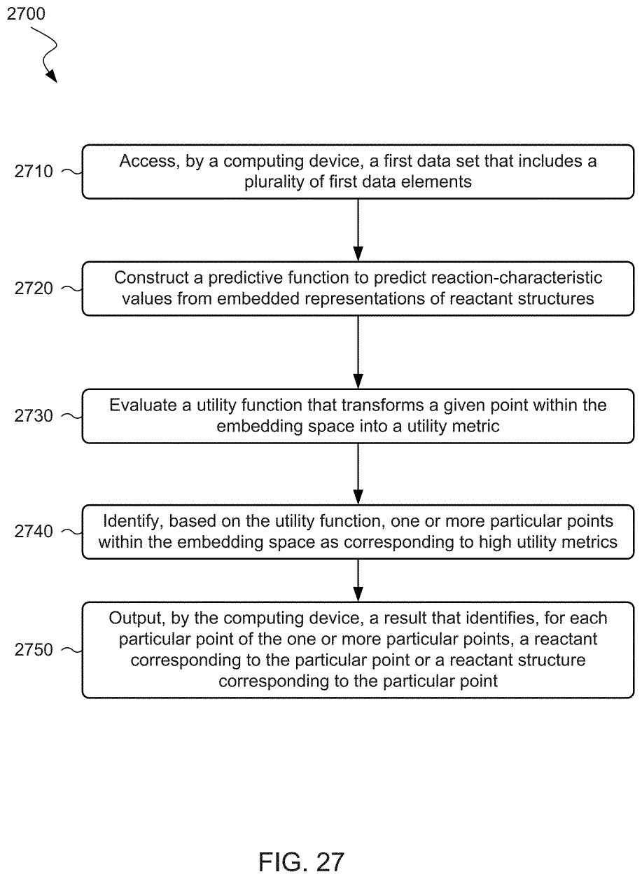

FIG. 27 is a flowchart of an example process 2700 associated with ionic liquid based depolymerization optimization.

At block 2710, process 2700 may include accessing a first data set that includes a plurality of first data elements. Each of the plurality of first data elements may characterize a depolymerization reaction. Each first data element may include an embedded representation of a structure of a reactant of the depolymerization and a reaction-characteristic value that characterizes a reaction between the reactant and a particular polymer. The embedded representation of the structure of the reactant may be identified as a set of coordinate values within an embedding space. For example, the embedding space may be encoded from SMILES or BSP representations and/or may be any embedding space described herein. The embedding space may capture at least 70%, at least 80%, at least 90%, at least 95%, at least 99%, or 100% of the relative variance.

In some embodiments, the embedding space may use principal components determined from PCA. SMILES, BSP, or other representations may be converted into descriptors, which can provide property information based on structure. As an example, mordred descriptors with a fixed length of 1,000 to 1,500, 1,500 to 2,000, or 2,000 to 3,000 may be used. A process feature matrix may then be reduced to remove duplicate entries and empty entries. In some embodiments, a value of 0 may be substituted for empty entries. A feature matrix may be obtained. The feature matrix may be normalized. The feature matrix may be decorrelated by removing columns that have a pairwise correlation coefficient greater than a predefined threshold (e.g., 0.4, 0.5, 0.6, 0.7, 0.8, or 0.9) and/or less than a negative of the predefined threshold. PCA may be used on the resulting feature matrix. PCA may result in 3, 4, 5, 5 to 10, 10 to 15, 15 to 20, 20 to 30, or more than 30 principal components (PCs).

The embedded representation may include a property of a reactant of a set of reactants in the depolymerization reaction. A property of the reactant may include viscosity, activity coefficient, bond type, enthalpy of formation, heat of combustion, or properties derived therefrom. The reactants in the plurality of first data elements may include ionic liquids, including any described herein. In some embodiments, the reactants may also include a solvent, including any described herein, and/or the polymer to be depolymerized. A suitable database, such as the National Institute of Standards and Technology (NIST) database for binary ionic liquids with known viscosities may be queried to provide property information.

The first data set may be seed data for Bayesian optimization. The first data elements may include variables associated with describing a depolymerization reaction.

The reaction-characteristic values may characterize an output of the depolymerization reaction. Outputs may include yield, amounts of products, conversion of products, selectivity, and/or profit. Outputs may not be known before running an experiment using reaction inputs but known after running the experiment. A computing device may access the first data set.

In some embodiments, the plurality of first data elements may include reaction input values that characterize an operating condition of the depolymerization reaction. For example, the reaction input variables may include time, temperature, ratio (e.g., ionic liquid to solvent), amount, cost, and/or pressure. The plurality of first data elements may also include a representation, which may or may not be embedded, of the solvent.

In some embodiments, the first data set may be generated from experiments using candidate molecules determined as described herein. For example, the candidate molecules may be determined using an embedding space and a variational autoencoder described herein.

At block 2720, process 2700 may include constructing a predictive function to predict reaction-characteristic values from embedded representations of reactant structures. Constructing the predictive function may use the first data set. In terms of Bayesian optimization, the predictive function may be an objective function, a black box function, a surrogate function, or a Gaussian Process prior. The function may have several setpoints for the first input data set with a probability distribution for the second input data set, similar to the graph shown for posterior distribution 2616 in FIG. 26. The predictive function may be constructed at least in part using training data corresponding to a set of molecules that were selected using Bayesian optimization. The computing device may construct the function.

Constructing the predictive function may include estimating reaction-characteristic values and/or reaction inputs not present in the first data set. These reaction-characteristic values and/or the reaction input values not present in the first data set may be discrete values even if the parameters are continuous (e.g., time, temperature, ratio, amount, embedding space). The estimated reaction-characteristic values and/or the reaction input values may be determined by a Gaussian Process.

The one or more particular points may be predefined discrete points. For example, the particular points may not include any value in the embedding space, but instead, the one or more particular points may be limited to only certain values in the embedding space. These values in the embedding space may correspond to molecules (e.g., ionic liquids) that are physically present in inventory at a site or available to be tested. Predefined may refer to the one or more particular points being determined before the predictive function is constructed or the utility function is evaluated. In some embodiments, the one or more particular point of the one or more particular points is not the same as any set of coordinate values within the embedding space in the plurality of first data elements.

At block 2730, process 2700 may include evaluating a utility function. The utility function may transform a given point within the embedding space into a utility metric that represents a degree to which identifying an experimentally derived reactant-characteristic value for the given point is predicted to improve the accuracy of the reactant-characteristic value. The utility function may be evaluated by evaluating an acquisition function. The acquisition function may minimize regret over the course of the optimization. The acquisition function may be GP Upper Confidence Bound (UCB). The acquisition function may be any acquisition function described herein. The utility function may be a concrete form of the acquisition function in which all optimization hyperparameters have been set. The utility function may include parameters for the degree of exploration and exploitation. The utility function may be any utility function described herein. Graph 2628 illustrates a utility function. The computing device may evaluate the utility function.

At block 2740, process 2700 may identify, based on the utility function, one or more particular points within the embedding space as corresponding to high utility metrics. For example, in FIG. 26, graph 2628 shows a utility function with star 2632 indicating the maximum. The values associated with that maximum may be one point of the one or more particular points.

At block 2750, process 2700 may include outputting, by the computing device, a result that identifies, for each particular point of the one or more particular points, a reactant corresponding to the particular point or a reactant structure corresponding to the particular point. The result may be displayed to a user.

In some embodiments, identifying the one or more particular points further comprises identifying one or more reaction-characteristic values as corresponding to the high utility metrics. The output may include an experimental procedure including the one or more reaction-characteristic values. For instance, the output may include reaction conditions. In some embodiments, process 2700 may include performing the experiment with the identified reaction-characteristic-values and/or reaction input values.

In some embodiments, process 2700 includes accessing an inventory data store including amounts of the reactants. For example, the inventory data store may include the amount of an ionic liquid or solvent. The amounts may be adjusted using the one or more particular points to determine an adjusted amount. For example, the one or more particular points may correspond to using n kilograms of ionic liquid A; process 2700 includes subtracting n kilograms of A from the amount of A in the inventory data store. Process 2700 may include comparing the adjusted amounts to a threshold value. The threshold value may be 0 or may be some amount reflecting a minimum amount to have readily available for experiments. An order may be outputted for additional amounts of the reactant when the adjusted amount is below the threshold value. The order may be a message sent from the computing device to another computing device that manages purchases of reactants.

In some embodiments, process 2700 may include determining that the one or more particular points are equivalent to one or more coordinate values of the set of coordinate values within the embedding space in the plurality of first data elements. Process 2700 may further include outputting a message conveying that the one or more particular points represent a converged solution. The computing device may end the Bayesian optimization. For example, the utility function may not be evaluated again.

In some embodiments, the first data set may include a point previously identified using the utility function. An experiment may have been performed using the point previously identified using the utility function to determine at least one reaction-characteristic value in the plurality of first data elements.

In some embodiments, the method may include updating the predictive function using a second data set. The second data set may include a plurality of second data elements. The second data elements may include the same data elements as the first data elements. A portion of the plurality of second data elements may be determined from performing an experiment using the reactant or the reactant structure identified by the outputted result.

Process 2700 may include additional implementations, such as any single implementation or any combination of implementations described below and/or in connection with one or more other processes described elsewhere herein.

Although FIG. 27 shows example blocks of process 2700, in some implementations, process 2700 may include additional blocks, fewer blocks, different blocks, or differently arranged blocks than those depicted in FIG. 27. Additionally, or alternatively, two or more of the blocks of process 2700 may be performed in parallel.

Embodiments may also include a depolymerization product resulting from performing an experiment with reactants and reaction-characteristic values and/or reaction input values corresponding to the one or more particular points identified by process 2700.

Embodiments may also include a method of performing an experiment. The method may include performing the experiment with reactants and/or reaction input values corresponding to the one or more particular points identified by process 2700.

Embodiments may also include a method of acquiring a reactant, including an ionic liquid, and/or a solvent. The identity of the reactant or solvent and/or the amount of the reactant or solvent may be determined by the reactants and/or reaction input values corresponding to the one or more particular points identified by process 2700.

Embodiments may also include a reactant and/or a solvent that is acquired after being identified by process 2700.

VI.D. Example Implementations

An approach for reducing dimensionality for ionic liquids for Bayesian optimization is described. Additionally, three examples of using the reduced dimensionality and applying a discrete sampling approach to Bayesian optimization via GP are provided. The enthalpy of mixing is minimized across mole fractions and chemical space. Additionally, the Bayesian optimization approach is tested with real depolymerization experiments, the results show that the process works well to predict polylactic acid (PLA) conversion and yield.

VI.D.1. Dimensionality Reduction

An example of dimensionality reduction is described. Physical property information (e.g., viscosity) regarding ionic liquids may be accessed. For example, pyilt2report (wgserve.de/pyilt2/pyilt2report.html) (accessed Sep. 15, 2021) was used to query the National Institute of Standards and Technology (NIST) for all binary ionic liquids (ILs) with known viscosities.

Cations and anions in the ionic liquids were converted to SMILES representations by querying a lookup table such as the Power User Gateway (PUG) web interface on the National Institutes of Health (NIH).

SMILES representations were converted into mordred descriptors, (github.com/mordred-descriptor/mordred) (accessed Sep. 15, 2021) each of which was a vector of fixed length of 1,826. Anion mordred descriptors were appended to cation mordred descriptors for binary ILs. NaN (not a number) values were filled with 0. A rectangular feature matrix of shape 117×3,652 was created. The 3,652 length is twice the fixed length of 1,826 as a result of appending vectors for both the cation and the anion. The 117 is the number of binary ILs in the data set.

The feature matrix may be reduced in size. For each of the 3,652 columns, their corresponding mean was subtracted and divided by n−1 (e.g., 116). This normalization may avoid columns appearing to be significant simply because the magnitudes of their values are higher than other columns. The feature matrix was decorrelated by computing pairwise correlation of columns and removing any columns that have correlation coefficient >0.5 or correlation coefficient <−0.5. The feature matrix included column indices that are duplicates based on high correlation, and one of each of the duplicate columns was retained. Each column was taken in order, and the column's correlation with every other column was computed. Any of these other columns that have a high correlation are removed. The process of analyzing correlation and removing columns is repeated for remaining columns. In this example, after decorrelation, the feature matrix has shape 117×26.

Principal Component Analysis (PCA) was carried out on this processed feature matrix. The first four principal components (PCs) captured more than 95% of the relative variance. These four principal components were used for Bayesian optimization.

In summary, four pieces of information were gathered to be used to facilitate transformations of chemical space: (1) means of columns in the binary IL dataset for use in normalizing columns; (2) binary samples accessible in a database; (3) highly correlated columns from the combined cation and anion descriptors; and (4) principal components from a feature matrix after decorrelation.

The reduced dimensionality provided by the PCs was used in a Bayesian optimization process. All observables (e.g., x_{obs} and xₛ in FIG. 26) were stored in arrays called rxnData and rxnDataResults. Columns corresponding to the four PCs (ilPC1, ilPC2, ilPC3, ilPC4) are included in rxnData. In addition, rxnData had more variables than just choice of IL, such as choice of solvent. More generally, the columns of rxnData used in the Gaussian Process (GP) are used in the Bayesian optimization. Similarly, rxnDataResults can have more than one target column (e.g., yield and conversion); the first target value was used for training.

The open-source BayesianOptimization (github.com/fmfn/BayesianOptimization) (accessed Sep. 15, 2021) package available via the MIT License may be used. The package was adapted for custom input of the arrays.

The Gaussian Process prior was constructed as described herein. The Matern kernel with v=2.5 was used, which is a standard choice for representing smoothly varying (i.e., twice-differentiable) functions. The posterior is constructed (e.g., as described with posterior distribution 2616). The next experiment is determined. The posterior along with a specified acquisition function were used to construct the utility function, U(xₛ), which is a concrete form of the acquisition function in which all optimization hyperparameters have been set. The acquisition function used was the GP Upper Confidence Bound (UCB), which minimizes regret over the course of optimization:

a_{UCB}(xₛ;{x_{obs},y_{obs}},θ)=μ(xₛ;{x_{obs},y_{obs}},θ)+κσ(xₛ;{x_{obs},y_{obs}},θ).

The utility function had parameters κ=2.576, xᵢ=0.

VI.D.2. Minimization of Mixing Enthalpy Across Mole Fraction and Temperature

Bayesian optimization was applied to determine the lowest enthalpy of mixing ionic liquid and solvent pairs. The PCs for ionic liquids determined above were used for Bayesian optimization. The lowest enthalpy of mixing indicates that the mixing is the most favored. Data on mixing enthalpies of all binary mixtures in the NIST database was collected. The data amounts to 214 IL and solvent pairs, with each pair having varying amounts of data for mixing enthalpy as a function of solvent mole fraction and temperature. To first order, the mixing enthalpy can be written as a regular solution mixing model: Hₘᵢₓ=Ωx_{A}x_{B} where Ω, a mixing parameter, disfavors mixing when Ω>0 and favors mixing when β<0. Since we have a binary system, x_{A}+x_{B}=1 so, to first order, the relationship between mixing enthalpy vs. mole fraction is quadratic.

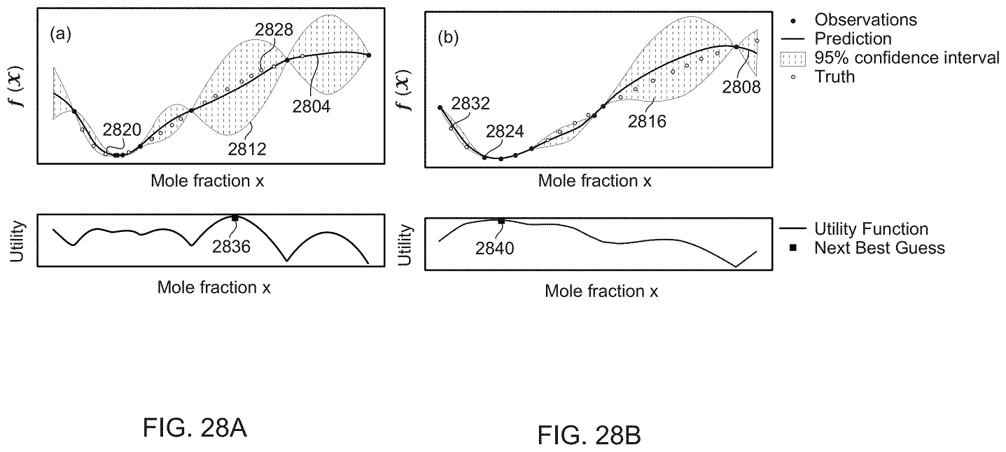

Minimization of enthalpy when Ω<0 is studied because the Ω>0 case is not as interesting (the minimum occurs at x=0 or x=1.0). FIGS. 28A and 28B show the result of applying Bayesian optimization to the minimization of Hₘᵢₓ over solvent mole fraction x_{obs}.

FIGS. 28A and 28B show two sampling approaches, both with the same utility function. The x-axis shows the mole fraction. The y-axis of the top graphs shows the black box function. Dashed lines 2804 and 2808 show the mean for the black box function. Shaded teal regions (e.g., regions 2812 and 2816) show 3σ for the black box function. The observations xₛ (e.g., points 2820 and 2824) are in red and the observables x_{obs} (e.g., points 2828 and 2832) are green. The yellow stars (e.g., stars 2836 and 2850) are x_{probe}, points that maximize the utility function.

In FIG. 28A, the minimization was done over continuous space, evidenced by the smoothly varying evaluation of f(x) for all values of mole fraction x. At the end of 10 iterations, the Bayesian optimization did not converge to the solution because the utility function suggests a next experiment (i.e., mole fraction corresponding to star 2836) that was away from the global minimum.

In FIG. 28B, the minimization was done over discrete space, evidenced by evaluations off(x) for only the given information, (i.e., the ground truth indicated by green points and the red points), and at the end of eight iterations converged to x=0.2052 which is the global minimum.

With this example, the enthalpy of mixing, as a black box function, was successfully demonstrated. The global minimum for enthalpy of mixing was found for Bayesian optimization with discrete sampling. On the other hand, by the end of 10 iterations in the continuous case, the mole fraction of x˜0.60 is suggested as a next experiment, which was far from the global minimum of x=0.2052 reached in the discrete case after eight iterations. Hence, discrete optimization was more efficient and cheaper than continuous optimization. This result is surprising because mole fraction is a continuous variable and not a discrete variable, and one would not expect that considering mole fraction as a discrete variable would result in a more efficient and cheaper optimization than considering mole fraction as a continuous variable.

VI.D.3. Minimization of Mixing Enthalpy Across Chemical Space

Bayesian optimization via GP across different molecules and chemistries was performed. The PCs for ionic liquids determined above were used for Bayesian optimization. Mixing enthalpy is minimized across all of chemical space for new ionic liquids and solvent pairs using only the discrete acquisition function. The black box function is now ƒ(xₛₒₗᵥₑₙₜ,T,IL,solvent)=Hₘᵢₓ, where xₛₒₗᵥₑₙₜ is the mole fraction of the solvent, T is the temperature, IL is the ionic liquid, solvent is the solvent, and Hₘᵢₓ is the enthalpy of mixing. The black box function cannot be written analytically even to first order.

The size of the seed experiments given to seed the Bayesian optimization was considered a “bundle.” Each “bundle” contained all the mole fraction and temperature data for a given ionic liquid and solvent pair versus Hₘᵢₓ. The bundle may contain similar information as the plurality of first data elements (in this example, ionic liquid, solvent, mole fraction) and the associated one or more output values (in this example, Hₘᵢₓ) of process 2700. Separating bundles by ionic liquid and solvent pairs and not by mole fractions is a realistic setting for seed experiments because the cost to purchase an ionic liquid to be used with a solvent is much greater than the cost to produce different mole fractions of solvent to IL. In other words, when an IL is purchased or created, the thermodynamic data is readily available or obtainable for a range of mole fractions.

Table 6 shows the 100 trial-averaged results of searching for the minimal enthalpy found with 5 single-additional experiments given a certain bundle size. Seed bundle sizes varied from 1 to 7. The average over 100 trials is shown, along with the average differential, or the improvement in the minimization, between the starting minimum enthalpy of mixing (in the bundle) and the final enthalpy of mixing. The greater the differential, the better the model was at suggesting successful experiments.

The bigger the bundle size, the more chemical and thermodynamic data was provided to the model to suggest experiments. Before any Bayesian optimization is performed, the average minimum enthalpy is expected to decrease with increasing initial seed bundle size because more data with more bundles are available.

Interestingly, in addition to decreasing average minimum enthalpy, the average differential (improvement based on Bayesian-optimized suggestions) also increased with a bigger bundle size, as shown in Table 6. Thus, with more chemical information provided as seed data, reactions with greater improvement were suggested.

Impressively, with four bundles and five additional experiments, the model searched across chemical and thermodynamic space and found conditions that resulted in mixing enthalpies lower than 97% of all other binary mixing enthalpies in the NIST database (n=4,672).

TABLE 6
Results for searching for minimal enthalpy
for different numbers of seed bundles

| No. of seed bundles | | | |
|---|---|---|---|
| (IL+ solvent pair | No. of | | |
| with all their T, | single | Avg. minimum | Avg. differential in |
| xₛₒₗᵥₑₙₜ | additional | found (kJ/mol) | ΔHₘᵢₓ (from initial |
| experiments) | exps. done | in 100 trials | bundle) |
| 1 | 5 | −0.851 | −1.401 |
| 2 | 5 | −1.327 | −2.553 |
| 3 | 5 | −1.6356 | −3.219 |
| 4 | 5 | −2.130 | −4.017 |
| 5 | 5 | −2.031 | −4.06 |
| 6 | 5 | −2.702 | −5.146 |
| 7 | 5 | −3.00 | −5.454 |

VI.D.4. Maximization of PLA Conversion and Yield

Bayesian optimization was applied to the depolymerization of polylactic acid (PLA). The PCs for ionic liquids determined above were used for Bayesian optimization. Conversion (C) and yield (Y) is the subject of optimization. Conversion is the amount of total product relative to amount of starting plastic, and yield is the amount of the target monomer relative to amount of starting plastic. The black box model is:

ƒ(x_{IL},IL,solvent,T,t)={C,Y}.

Experiments on the depolymerization of PLA (n_{obs}=94) were gathered from a literature review. These curated experiments served as x_{obs} for Bayesian optimization. Because the experiments represent a biased population (successful experiments are published, unsuccessful experiments are not), the Bayesian optimization is not subject to a realistic observation space. Thus, Bayesian optimization performance was compared with a baseline, random-draw scenario. The purely Bayesian approach (1 seed experiment and 5 Bayesian-optimized steps) was compared with the purely random approach (6 randomly selected experiments), averaged over 100 trials.

Each trial involved picking nSeed (number of seed experiments), which is a random draw out of the total curated 94 experiments. Then nExp (additional experiments) are suggested and “carried out” (in this case, looked up in rxnDataResults). The total number of experiments was kept constant. At the end of six experiments, the maximal conversion or yield was checked. If the conversion (yield) is greater than 95% (85%), the trial was a success.

Table 7 shows the results of doing 100 trials to predict conversion and yield. The 100 trial-averages from Bayesian optimization (nSeed/nExp=1/5) were compared to the random draw (nSeed/nExp=6/0). The results for conversion are on the left and the results for yield are on the right. The percent success (%) is the average likelihood that the maximized reaction is greater than 95% for conversion and greater than 85% for yield. The differential is the average difference between the maximum of nSeed and the maximum of the entire process (nSeed, nExp). Since there are no iterations in nSeed/nExp=6/0, the differential is 0%.

TABLE 7
Predicting conversion and yield with
different seed and experiment numbers

| | Percent | Conversion | | Percent | Yield |
|---|---|---|---|---|---|
| | success | differential | | success | differ- |
| nSeed/nExp | (%) | (%) | nSeed/nExp | (%) | ential (%) |
| 1/5 (purely | 100 | −24.3 | 1/5 (purely | 99 | −28.03 |
| Bayesian) | | | Bayesian) | | |
| 2/4 | 96 | −9.9 | 2/4 | 89 | −10.1 |
| 3/3 | 91 | −5.9 | 3/3 | 94 | −6.9 |
| 4/2 | 95 | −0.83 | 4/2 | 90 | −2.9 |
| 5/1 | 85 | −0.52 | 5/1 | 91 | −0.43 |
| 6/0 (purely | 90 | 0 | 6/0 (purely | 94 | 0 |
| random) | | | random) | | |

Based on these results, the purely Bayesian approach was the most successful approach (100% success rate for conversion, 99% success rate for yield), and the Bayesian model outperformed the random-draw in spite of the biased dataset.

VI.D.5. Depolymerization of Mixed Waste

Bayesian optimization approaches are applied to reactions to depolymerize mixed waste (such as PET/PLA mixtures), contaminated waste (real-world challenges expected to reduce conversion and yield), and critically overlooked waste streams (black plastics) that have yet to be addressed in academic research. An embedding space of the ionic liquids described herein is used in optimizing the depolymerization reactions.

### VII. System Environment

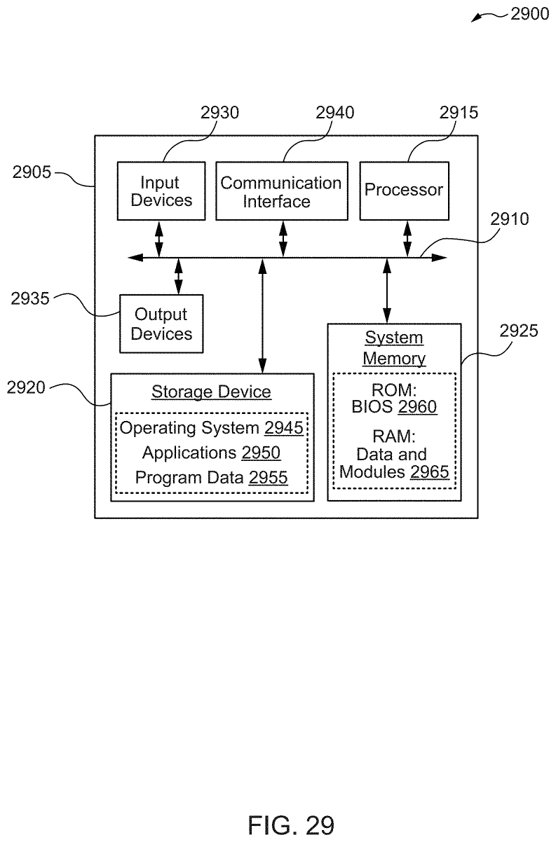

FIG. 29 is an illustrative architecture of a computing system 2900 implemented as some embodiments of the present disclosure. The computing system 2900 is only one example of a suitable computing system and is not intended to suggest any limitation as to the scope of use or functionality of the present disclosure. Also, computing system 2900 should not be interpreted as having any dependency or requirement relating to any one or combination of components illustrated in computing system 2900.

As shown in FIG. 29, computing system 2900 includes a computing device 2905. The computing device 2905 can be resident on a network infrastructure such as within a cloud environment, or may be a separate independent computing device (e.g., a computing device of a service provider). The computing device 2905 may include a bus 2910, processor 2915, a storage device 2920, a system memory (hardware device) 2925, one or more input devices 2930, one or more output devices 2935, and a communication interface 2940.

The bus 2910 permits communication among the components of computing device 2905. For example, bus 2910 may be any of several types of bus structures including a memory bus or memory controller, a peripheral bus, and a local bus using any of a variety of bus architectures to provide one or more wired or wireless communication links or paths for transferring data and/or power to, from, or between various other components of computing device 2905.

The processor 2915 may be one or more processors, microprocessors, or specialized dedicated processors that include processing circuitry operative to interpret and execute computer readable program instructions, such as program instructions for controlling the operation and performance of one or more of the various other components of computing device 2905 for implementing the functionality, steps, and/or performance of the present disclosure. In certain embodiments, processor 2915 interprets and executes the processes, steps, functions, and/or operations of the present disclosure, which may be operatively implemented by the computer readable program instructions. For example, processor 2915 can retrieve, e.g., import and/or otherwise obtain or generate ionic liquid properties, encode molecule information into an embedding space, decode a point in an embedding space into a molecule, construct predictive functions, and evaluate utility functions. In embodiments, the information obtained or generated by the processor 2915 can be stored in the storage device 2920.

The storage device 2920 may include removable/non-removable, volatile/non-volatile computer readable media, such as, but not limited to, non-transitory machine readable storage medium such as magnetic and/or optical recording media and their corresponding drives. The drives and their associated computer readable media provide for storage of computer readable program instructions, data structures, program modules and other data for operation of computing device 2905 in accordance with the different aspects of the present disclosure. In embodiments, storage device 2920 may store operating system 2945, application programs 2950, and program data 2955 in accordance with aspects of the present disclosure.

The system memory 2925 may include one or more storage mediums, including for example, non-transitory machine readable storage medium such as flash memory, permanent memory such as read-only memory (“ROM”), semi-permanent memory such as random access memory (“RAM”), any other suitable type of non-transitory storage component, or any combination thereof. In some embodiments, an input/output system 2960 (BIOS) including the basic routines that help to transfer information between the various other components of computing device 2905, such as during start-up, may be stored in the ROM. Additionally, data and/or program modules 2965, such as at least a portion of operating system 2945, program modules, application programs 2950, and/or program data 2955, that are accessible to and/or presently being operated on by processor 2915, may be contained in the RAM. In embodiments, the program modules 2965 and/or application programs 2950 can comprise, for example, a processing tool to identify and annotate spectrum data, a metadata tool to append data structures with metadata, and one or more encoder networks and/or encoder-decoder networks to predict spectra, which provides the instructions for execution of processor 2915.

The one or more input devices 2930 may include one or more mechanisms that permit an operator to input information to computing device 2905, including, but not limited to, a touch pad, dial, click wheel, scroll wheel, touch screen, one or more buttons (e.g., a keyboard), mouse, game controller, track ball, microphone, camera, proximity sensor, light detector, motion sensors, biometric sensor, and combinations thereof. The one or more output devices 2935 may include one or more mechanisms that output information to an operator, such as, but not limited to, audio speakers, headphones, audio line-outs, visual displays, antennas, infrared ports, tactile feedback, printers, or combinations thereof.

The communication interface 2940 may include any transceiver-like mechanism (e.g., a network interface, a network adapter, a modem, or combinations thereof) that enables computing device 2905 to communicate with remote devices or systems, such as a mobile device or other computing devices such as, for example, a server in a networked environment, e.g., cloud environment. For example, computing device 2905 may be connected to remote devices or systems via one or more local area networks (LAN) and/or one or more wide area networks (WAN) using communication interface 2940.

As discussed herein, computing system 2900 may be configured to train an encoder-decoder network to predict an characteristic spectral features from a structural representation of a material obtained as a structure string. In particular, computing device 2905 may perform tasks (e.g., process, steps, methods and/or functionality) in response to processor 2915 executing program instructions contained in non-transitory machine readable storage medium, such as system memory 2925. The program instructions may be read into system memory 2925 from another computer readable medium (e.g., non-transitory machine readable storage medium), such as data storage device 2920, or from another device via the communication interface 2940 or server within or outside of a cloud environment. In embodiments, an operator may interact with computing device 2905 via the one or more input devices 2930 and/or the one or more output devices 2935 to facilitate performance of the tasks and/or realize the end results of such tasks in accordance with aspects of the present disclosure. In additional or alternative embodiments, hardwired circuitry may be used in place of or in combination with the program instructions to implement the tasks, e.g., steps, methods and/or functionality, consistent with the different aspects of the present disclosure. Thus, the steps, methods and/or functionality disclosed herein can be implemented in any combination of hardware circuitry and software.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

All patents, patent applications, publications, and descriptions mentioned herein are hereby incorporated by reference in their entirety for all purposes as if each individual publication or patent were specifically and individually indicated to be incorporated by reference and are incorporated herein by reference to disclose and describe the methods and/or materials in connection with Which the publications are cited. None is admitted to be prior art.

What is claimed is:

1. A computer-implemented method comprising:
accessing a multi-dimensional embedding space that supports relating embeddings of molecules to predicted values of a given property of the molecules;
identifying one or more points of interest within the multi-dimensional embedding space based on the predicted values, wherein each of the one or more points of interest:
includes a set of coordinate values within the multi-dimensional embedding space,
conveys spatial information of atoms or bonds in the molecule, and
is associated with a corresponding predicted value of the given property;
generating, for each of the one or more points of interest, a structural representation of a molecule by transforming the set of coordinate values included in the point of interest using a decoder network, wherein training of the decoder network included learning to transform positions within the embedding space to outputs representing molecular-structure characteristics, wherein the training of the decoder network was performed at least in part concurrently to training an encoder network; and
outputting a result that identifies, for each of the one or more points of interest, the structural representation of the molecule corresponding to the point of interest.

2. The method of claim 1, wherein training of the encoder network included learning to transform partial or complete bond string and position (BSP) representations of molecules into positions within the embedding space, and wherein each BSP representation identifies relative positions of atoms connected by a bond in the represented molecule.

3. The method of claim 2, wherein each BSP representation of the molecules used to train the encoder network includes a set of coordinates for each of the atoms connected by the bond in the represented molecule and further identifies each of the atoms connected by the bond in the represented molecule.

4. The method of claim 2, wherein the BSP representation of the molecules used to train the encoder network to identify, for each of at least some bonds in a respective molecule, a bond type.

5. The method of claim 2, wherein a format of the structural representation identified in the result is different than the BSP representation.

6. The method of claim 1, wherein training of the encoder network included learning to transform partial or complete molecular graph representations of molecules into positions within the embedding space, and wherein each molecular graph representation identifies angles and distances of bonds in the represented molecule.

7. The method of claim 1, wherein the decoder network and the encoder network were trained by training a transformer model that uses self-attention, wherein the transformer model includes the decoder network and the encoder network.

8. The method of claim 1, wherein the decoder network and the encoder network were trained by training a transformer model that includes an attention head.

9. The method of claim 1, further comprising training a machine-learning model that includes the encoder network and the decoder network by:
accessing a set of supplemental training elements, wherein each of the set of training elements includes a representation of a structure of a corresponding given molecule;
masking, for each supplemental training element in the set of supplemental training elements, at least part of the representation to obscure at least part of the structure of the corresponding given molecule; and
training the machine-learning model to predict the obscured at least part of the structure.

10. The method of claim 1, wherein training of the encoder network further included fine-tuning the encoder network to transform positions within the space to predictions corresponding to values of the given property.

11. A system comprising:
one or more data processors; and
a non-transitory computer readable storage medium containing instructions which, when executed on the one or more data processors, cause the one or more data processors to perform a method comprising:
accessing a multi-dimensional embedding space that supports relating embeddings of molecules to predicted values of a given property of the molecules;
identifying one or more points of interest within the multi-dimensional embedding space based on the predicted values, wherein each of the one or more points of interest:
includes a set of coordinate values within the multi-dimensional embedding space,
conveys spatial information of atoms or bonds in the molecule, and
is associated with a corresponding predicted value of the given property;
generating, for each of the one or more points of interest, a structural representation of a molecule by transforming the set of coordinate values included in the point of interest using a decoder network, wherein training of the decoder network included learning to transform positions within the embedding space to outputs representing molecular-structure characteristics, wherein the training of the decoder network was performed at least in part concurrently to training an encoder network; and
outputting a result that identifies, for each of the one or more points of interest, the structural representation of the molecule corresponding to the point of interest.

12. The system of claim 11, wherein training of the encoder network included learning to transform partial or complete bond string and position (BSP) representations of molecules into positions within the embedding space, and wherein each BSP representation identifies relative positions of atoms connected by a bond in the represented molecule.

13. The system of claim 12, wherein each BSP representation of the molecules used to train the encoder network includes a set of coordinates for each of the atoms connected by the bond in the represented molecule and further identifies each of the atoms connected by the bond in the represented molecule.

14. The system of claim 12, wherein the BSP representation of the molecules used to train the encoder network to identify, for each of at least some bonds in a respective molecule, a bond type.

15. The system of claim 12, wherein a format of the structural representation identified in the result is different than the BSP representation.

16. The system of claim 11, wherein training of the encoder network included learning to transform partial or complete molecular graph representations of molecules into positions within the embedding space, and wherein each molecular graph representation identifies angles and distances of bonds in the represented molecule.

17. The system of claim 11, wherein the decoder network and the encoder network were trained by training a transformer model that uses self-attention, wherein the transformer model includes the decoder network and the encoder network.

18. The system of claim 11, wherein the decoder network and the encoder network were trained by training a transformer model that includes an attention head.

19. The system of claim 11, the method further comprising training a machine-learning model that includes the encoder network and the decoder network by:
accessing a set of supplemental training elements, wherein each of the set of training elements includes a representation of a structure of a corresponding given molecule;
masking, for each supplemental training element in the set of supplemental training elements, at least part of the representation to obscure at least part of the structure of the corresponding given molecule; and
training the machine-learning model to predict the obscured at least part of the structure.

20. A computer-program product tangibly embodied in a non-transitory machine-readable storage medium, including instructions configured to cause one or more data processors to perform a method comprising:
accessing a multi-dimensional embedding space that supports relating embeddings of molecules to predicted values of a given property of the molecules;
identifying one or more points of interest within the multi-dimensional embedding space based on the predicted values, wherein each of the one or more points of interest:
includes a set of coordinate values within the multi-dimensional embedding space,
conveys spatial information of atoms or bonds in the molecule, and
is associated with a corresponding predicted value of the given property;
generating, for each of the one or more points of interest, a structural representation of a molecule by transforming the set of coordinate values included in the point of interest using a decoder network, wherein training of the decoder network included learning to transform positions within the embedding space to outputs representing molecular-structure characteristics, wherein the training of the decoder network was performed at least in part concurrently to training an encoder network; and
outputting a result that identifies, for each of the one or more points of interest, the structural representation of the molecule corresponding to the point of interest.
